SEQUENCE LISTING (38 sequences)

SEQ ID NO: 1 - protein, 6 aa, Artificial Sequence
  Description of Artificial Sequence Synthetic peptide
QSISSY

SEQ ID NO: 2 - protein, 3 aa, Artificial Sequence
  Description of Artificial Sequence Synthetic peptide
NAA

SEQ ID NO: 3 - protein, 9 aa, Artificial Sequence
  Description of Artificial Sequence Synthetic peptide
QQAATYPAT

SEQ ID NO: 4 - protein, 8 aa, Artificial Sequence
  Description of Artificial Sequence Synthetic peptide
GFTFSSYA

SEQ ID NO: 5 - protein, 8 aa, Artificial Sequence
  Description of Artificial Sequence Synthetic peptide
IAYGGGAT

SEQ ID NO: 6 - protein, 9 aa, Artificial Sequence
  Description of Artificial Sequence Synthetic peptide
AKGLSNFDY

SEQ ID NO: 7 - protein, 107 aa, Artificial Sequence
  Description of Artificial Sequence Synthetic polypeptide
DIQMTQSPSSLSASVGDRVTITCRASQSISSYLNWYQQKPGKAPKLLIYNAAFLYSGVPSRFSGSRSGTDFTLTISSLQPEDFATYYCQQAATYPATFGQGTKVEIK

SEQ ID NO: 8 - protein, 116 aa, Artificial Sequence
  Description of Artificial Sequence Synthetic polypeptide
EVQLVESGGGLVQPGGSLRLSCAASGFTFSSYAIHWVRQAPGKGLEWVARIAYGGGATRYADSVKGRFTISADTSKNTAYLQMNSLRAEDTAVYYCAKGLSNFDYWGQGTLVTVSS

SEQ ID NO: 9 - protein, 5 aa, Artificial Sequence
  Description of Artificial Sequence Synthetic peptide
SSVSY

SEQ ID NO: 10 - protein, 3 aa, Artificial Sequence
  Description of Artificial Sequence Synthetic peptide
STS

SEQ ID NO: 11 - protein, 9 aa, Artificial Sequence
  Description of Artificial Sequence Synthetic peptide
QQRSSYPPT

SEQ ID NO: 12 - protein, 8 aa, Artificial Sequence
  Description of Artificial Sequence Synthetic peptide
GYTFTDYV

SEQ ID NO: 13 - protein, 8 aa, Artificial Sequence
  Description of Artificial Sequence Synthetic peptide
IYPGNNTT

SEQ ID NO: 14 - protein, 21 aa, Artificial Sequence
  Description of Artificial Sequence Synthetic peptide
ARESLGIYYGSTYKGLYAMDY

SEQ ID NO: 15 - protein, 108 aa, Artificial Sequence
  Description of Artificial Sequence Synthetic polypeptide
QIVLTQSPAIMSASPGEKVTITCSASSSVSYMHWFQQKPGTSPKLWIYSTSNLASGVPARFSGSGSGTSYSLTISRMEAEDAATYYCQQRSSYPPTFGAGTKLELKRT

SEQ ID NO: 16 - protein, 128 aa, Artificial Sequence
  Description of Artificial Sequence Synthetic polypeptide
QVQLQQSGPELVKPGASVKMSCKASGYTFTDYVISWVKQRTGQGLEWIGEIYPGNNTTYYNDNFRGKATLTVDKSSSTAYMQLSSLTSEDSAVYYCARESLGIYYGSTYKGLYAMDYWGQGTTLTVSS

SEQ ID NO: 17 - protein, 8 aa, Artificial Sequence
  Description of Artificial Sequence Synthetic peptide
GIIFSDYR

SEQ ID NO: 18 - protein, 7 aa, Artificial Sequence
  Description of Artificial Sequence Synthetic peptide
ISTGGSI

SEQ ID NO: 19 - protein, 5 aa, Artificial Sequence
  Description of Artificial Sequence Synthetic peptide
NAGLR

SEQ ID NO: 20 - protein, 113 aa, Artificial Sequence
  Description of Artificial Sequence Synthetic polypeptide
MAQVQLQESGGGLVQAGGSLRLTCVASGIIFSDYRVAWYRQAPGKQRELVARISTGGSILYADSVKGRFTISRDNAKNTVNLQMNSLKPEDTAVYYCNAGLRWGQGTQVTVSS

SEQ ID NO: 21 - protein, 8 aa, Artificial Sequence
  Description of Artificial Sequence Synthetic peptide
GRTFSRAT

SEQ ID NO: 22 - protein, 8 aa, Artificial Sequence
  Description of Artificial Sequence Synthetic peptide
IRWSTEST

SEQ ID NO: 23 - protein, 20 aa, Artificial Sequence
  Description of Artificial Sequence Synthetic peptide
ATDLSTYYGAIDTGADDYDY

SEQ ID NO: 24 - protein, 129 aa, Artificial Sequence
  Description of Artificial Sequence Synthetic polypeptide
MAQVTLKESGGGLVQAGGSLRLSCAVSGRTFSRATMAWFRQAPGKEREFVAGIRWSTESTYYADSVKGRFTISRDRAKNTVYLDMNSLKPEDTAAYYCATDLSTYYGAIDTGADDYDYWGQGTQVTVSS

SEQ ID NO: 25 - protein, 8 aa, Artificial Sequence
  Description of Artificial Sequence Synthetic peptide
GRISTTYA

SEQ ID NO: 26 - protein, 8 aa, Artificial Sequence
  Description of Artificial Sequence Synthetic peptide
IYWSGGMT

SEQ ID NO: 27 - protein, 23 aa, Artificial Sequence
  Description of Artificial Sequence Synthetic peptide
AADPRAAYYYGTSDYTLPGRYNN

SEQ ID NO: 28 - protein, 132 aa, Artificial Sequence
  Description of Artificial Sequence Synthetic polypeptide
MAQVQLVQSGGGLVQPGGSLRLSCAASGRISTTYAMAWFRQAPGKEREFVAAIYWSGGMTKYADSVKGRSTISRDNAKNTVLLQMNSLKSGDTAVYYCAADPRAAYYYGTSDYTLPGRYNNWGQGTQVTVSS

SEQ ID NO: 29 - protein, 8 aa, Artificial Sequence
  Description of Artificial Sequence Synthetic peptide
GSISSFEQ

SEQ ID NO: 30 - protein, 7 aa, Artificial Sequence
  Description of Artificial Sequence Synthetic peptide
FRSDGST

SEQ ID NO: 31 - protein, 9 aa, Artificial Sequence
  Description of Artificial Sequence Synthetic peptide
NTYPVVLYN

SEQ ID NO: 32 - protein, 117 aa, Artificial Sequence
  Description of Artificial Sequence Synthetic polypeptide
MAQVTLKESGGGLVQPGGSLRLSCAASGSISSFEQMAWYRQAPGKQRALVARFRSDGSTKYTDSVKDRFTISRDNAKSTVYLQMNSLKPEDTAVYYCNTYPVVLYNWGQGTQVTVSS

SEQ ID NO: 33 - protein, 8 aa, Artificial Sequence
  Description of Artificial Sequence Synthetic peptide
GRTFSHAT

SEQ ID NO: 34 - protein, 8 aa, Artificial Sequence
  Description of Artificial Sequence Synthetic peptide
ITWSTEST

SEQ ID NO: 35 - protein, 20 aa, Artificial Sequence
  Description of Artificial Sequence Synthetic peptide
AADLSTYYGAIDTGADDYDY

SEQ ID NO: 36 - protein, 129 aa, Artificial Sequence
  Description of Artificial Sequence Synthetic polypeptide
MAQVTLQESGGGLVQAGGSLRLSCAASGRTFSHATMAWFRQAPGKEREFVARITWSTESTYYEYSVKGRFTISKDRAKNTVYLDMNRLKPEDTAAYYCAADLSTYYGAIDTGADDYDYWGQGTQVTVSS

SEQ ID NO: 37 - protein, 129 aa, Artificial Sequence
  Description of Artificial Sequence Synthetic polypeptide
MAQVQLQESGGGLVQAGGSLRLSCAVSGRTFSRATMAWFRQAPGKEREFVAGIRWSTESTYYADSVKGRFTISRDRAKNTVYLDMNSLKPEDTAAYYCATDLSTYYGAIDTGADDYDYWGQGTQVTVSS

SEQ ID NO: 38 - protein, 9 aa, Homo sapiens
VMAPRTLFL

(19) United States
(12) Patent Application Publication
(10) Pub. No.: US 2021/0253713 A1
(43) Pub. Date: Aug. 19, 2021

(54) ANTIBODIES TARGETING A COMPLEX COMPRISING NON-CLASSICAL HLA-I AND NEOANTIGEN AND THEIR METHODS OF USE

(71) Applicant: ABEXXA BIOLOGICS, INC., Arlington, TX (US)

(72) Inventor: Jon WEIDANZ, Arlington, TX (US)

(21) Appl. No.: 17/250,443

(22) PCT Filed: Jul. 23, 2019
(86) PCT No.: PCT/US2019/043108

§ 371 Date: Jan. 21, 2021

Related U.S. Application Data
(60) Provisional application No. 62/702,143, filed on Jul. 23, 2018.

(51) Int. Cl. C07K 16/28; A61K 47/68; A61P 35/00

(52) U.S. Cl. CPC C07K 16/2833; A61K 47/6849; A61P 35/00; A61K 2039/545; C07K 2317/34; C07K 2317/92; C07K 2317/565; C07K 2317/32

(57) ABSTRACT

Provided herein are antibodies that selectively bind to complex comprising a non-classical HLA-I (e.g. HL A-E) and a neoantigen (e.g. VMAPRTLFL (SEQ ID NO: 38)) having variable heavy chain domains (VH), variable light chain domains (VL), and complementarity determining regions (CDRs) as disclosed herein, as well as methods and uses thereof.

### CROSS-REFERENCE

This application claims the benefit of U.S. Provisional Application No. 62/702,143 filed Jul. 23, 2018, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Jul. 23, 2019, is named 50626-706_601_SL.txt and is 18,500 bytes in size.

### SUMMARY

Disclosed herein, are antibodies that selectively bind to a complex comprising a non-classical HLA-I (e.g. HLA-E) and a neoantigen, thereby modulating an immune response against cancer cells.

Disclosed herein, in certain embodiments, are monoclonal antibodies or an antigen-binding fragments thereof, comprising a light chain variable domain (VL) comprising an amino acid sequence at least 80% identical to an amino acid sequence set forth as SEQ ID NO: 7, or SEQ ID NO: 15. Disclosed herein, in certain embodiments, are monoclonal antibodies or an antigen-binding fragments thereof, comprising a light chain variable domain (VL) comprising an amino acid sequence at least 90% identical to an amino acid sequence set forth as SEQ ID NO: 7, or SEQ ID NO: 15. Disclosed herein, in certain embodiments, are monoclonal antibodies or an antigen-binding fragments thereof, comprising a light chain variable domain (VL) comprising an amino acid sequence at least 95% identical to an amino acid sequence set forth as SEQ ID NO: 7, or SEQ ID NO: 15. Disclosed herein, in certain embodiments, are monoclonal antibodies or an antigen-binding fragments thereof, comprising a light chain variable domain (VL) comprising an amino acid sequence at least 99% identical to an amino acid sequence set forth as SEQ ID NO: 7, or SEQ ID NO: 15. Disclosed herein, in certain embodiments, are monoclonal antibodies or an antigen-binding fragments thereof, comprising a light chain variable domain (VL) comprising an amino acid sequence 100% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a heavy chain variable domain (VH) comprising an amino acid sequence at least 90% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a heavy chain variable domain (VH) comprising an amino acid sequence at least 95% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a heavy chain variable domain (VH) comprising an amino acid sequence at least 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a heavy chain variable domain (VH) comprising an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof selectively binds to a complex comprising an HLA-E and a neoantigen. In some instances, the monoclonal antibody or antigen-binding fragment thereof does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL). In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL (SEQ ID NO: 38). In some instances, the monoclonal antibody or antigen-binding fragment thereof is a murine antibody, a chimeric antibody, a camelid antibody, a humanized antibody, or a human antibody. In some instances, the monoclonal antibody or antigen-binding fragment thereof is a TCR-like antibody. In some instances, the monoclonal antibody or antigen-binding fragment thereof is a multispecific antibody. In some instances, the monoclonal antibody or antigen-binding fragment thereof is a multifunctional antibody. In some instances, the monoclonal antibody or antigen-binding fragment thereof antibody further comprises a conjugated therapeutic moiety. In some instances, the selective binding of the antibody to the complex comprising the HLA-E and the neoantigen induces an immune response in a cell. In some instances, the immune response comprises activation of T cells. In some instances, the T cell is a CD8+ T cell. In some instances, the immune response comprises activation of cytotoxic T cells (CTLs). In some instances, the cell is a cancer cell.

Disclosed herein, in certain embodiments, are monoclonal antibodies or an antigen-binding fragments thereof, comprising a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. Disclosed herein, in certain embodiments, are monoclonal antibodies or an antigen-binding fragments thereof, comprising a heavy chain variable domain (VH) comprising an amino acid sequence at least 90% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. Disclosed herein, in certain embodiments, are monoclonal antibodies or an antigen-binding fragments thereof, comprising a heavy chain variable domain (VH) comprising an amino acid sequence at least 95% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. Disclosed herein, in certain embodiments, are monoclonal antibodies or an antigen-binding fragments thereof, comprising a heavy chain variable domain (VH) comprising an amino acid sequence at least 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. Disclosed herein, in certain embodiments, are monoclonal antibodies or an antigen-binding fragments thereof, comprising a heavy chain variable domain (VH) comprising an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a light chain variable domain (VL) comprising an amino acid sequence at least 90% identical to an amino acid sequence set forth as SEQ ID NO: 7, or SEQ ID NO: 15. In some instances, the monoclonal antibody or antigen-binding fragment thereof comprises a light chain variable domain (VL) comprising an amino acid sequence at least 95% identical to an amino acid sequence set forth as SEQ ID NO: 7, or SEQ ID NO: 15. In some instances, the monoclonal antibody or antigen-binding fragment thereof comprises a light chain variable domain (VL) comprising an amino acid sequence at least 99% identical to an amino acid sequence set forth as SEQ ID NO: 7, or SEQ ID NO: 15. In some instances, the monoclonal antibody or antigen-binding fragment thereof comprises a light chain variable domain (VL) comprising an amino acid sequence 100% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to SEQ ID NO: 8 and a light chain variable domain (VL) comprising an amino acid sequence at least 80% identical to SEQ ID NO: 7. In some instances, the monoclonal antibody or antigen-binding fragment thereof comprises a heavy chain variable domain (VH) comprising an amino acid sequence at least 80% identical to SEQ ID NO: 16 and a light chain variable domain (VL) comprising an amino acid sequence at least 80% identical to SEQ ID NO: 15. In some instances, the monoclonal antibody or antigen-binding fragment thereof selectively binds to a complex comprising an HLA-E and a neoantigen. In some instances, the monoclonal antibody or antigen-binding fragment thereof does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL). In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL (SEQ ID NO: 38). In some instances, the monoclonal antibody or antigen-binding fragment thereof is a murine antibody, a chimeric antibody, a camelid antibody, a humanized antibody, or a human antibody. In some instances, the monoclonal antibody or antigen-binding fragment thereof is a TCR-like antibody. In some instances, the monoclonal antibody or antigen-binding fragment thereof is a multispecific antibody. In some instances, the monoclonal antibody or antigen-binding fragment thereof is a multifunctional antibody. In some instances, the monoclonal antibody or antigen-binding fragment thereof antibody further comprises a conjugated therapeutic moiety. In some instances, the selective binding of the antibody to the complex comprising the HLA-E and the neoantigen induces an immune response in a cell. In some instances, the immune response comprises activation of T cells. In some instances, the T cell is a CD8+ T cell. In some instances, the immune response comprises activation of cytotoxic T cells (CTLs). In some instances, the cell is a cancer cell.

Disclosed herein, in certain embodiments, are monoclonal antibodies or an antigen-binding fragments thereof, comprising a light chain complementarity determining region (CDR) having an amino acid sequence at least 80% identical to at least one of the amino acid sequences set forth as SEQ ID NOS: 1-3, or 9-11. Disclosed herein, in certain embodiments, are monoclonal antibodies or an antigen-binding fragments thereof, comprising a light chain complementarity determining region (CDR) having an amino acid sequence at least 90% identical to at least one of the amino acid sequences set forth as SEQ ID NOS: 1-3, or 9-11. Disclosed herein, in certain embodiments, are monoclonal antibodies or an antigen-binding fragments thereof, comprising a light chain complementarity determining region (CDR) having an amino acid sequence at least 95% identical to at least one of the amino acid sequences set forth as SEQ ID NOS: 1-3, or 9-11. Disclosed herein, in certain embodiments, are monoclonal antibodies or an antigen-binding fragments thereof, comprising a light chain complementarity determining region (CDR) having an amino acid sequence at least 99% identical to at least one of the amino acid sequences set forth as SEQ ID NOS: 1-3, or 9-11. Disclosed herein, in certain embodiments, are monoclonal antibodies or an antigen-binding fragments thereof, comprising a light chain complementarity determining region (CDR) having an amino acid sequence 100% identical to at least one of the amino acid sequences set forth as SEQ ID NOS: 1-3, or 9-11. In some instances, the monoclonal antibody or antigen-binding fragment thereof comprises a heavy chain complementarity determining region (CDR) having an amino acid sequence at least 80% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a heavy chain complementarity determining region (CDR) having an amino acid sequence at least 90% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a heavy chain complementarity determining region (CDR) having an amino acid sequence at least 95% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a heavy chain complementarity determining region (CDR) having an amino acid sequence at least 99% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a heavy chain complementarity determining region (CDR) having an amino acid sequence 100% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15; and a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof selectively binds to a complex comprising an HLA-E and a neoantigen. In some instances, the monoclonal antibody or antigen-binding fragment thereof does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL). In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL (SEQ ID NO: 38). In some instances, the monoclonal antibody or antigen-binding fragment thereof is a murine antibody, a chimeric antibody, a camelid antibody, a humanized antibody, or a human antibody. In some instances, the monoclonal antibody or antigen-binding fragment thereof is a TCR-like antibody. In some instances, the monoclonal antibody or antigen-binding fragment thereof is a multispecific antibody. In some instances, the monoclonal antibody or antigen-binding fragment thereof is a multifunctional antibody. In some instances, the monoclonal antibody or antigen-binding fragment thereof antibody further comprises a conjugated therapeutic moiety. In some instances, the selective binding of the antibody to the complex comprising the HLA-E and the neoantigen induces an immune response in a cell. In some instances, the immune response comprises activation of T cells. In some instances, the T cell is a CD8+ T cell. In some instances, the immune response comprises activation of cytotoxic T cells (CTLs). In some instances, the cell is a cancer cell.

Disclosed herein, in certain embodiments, are monoclonal antibodies or an antigen-binding fragments thereof, comprising a heavy chain complementarity determining region (CDR) having an amino acid sequence at least 80% identical to at least one of the amino acid sequences set forth as SEQ ID NOS: 4-6, 12-14, 17-19, 21-23, 25-27, 29-31, and 33-35. Disclosed herein, in certain embodiments, are monoclonal antibodies or an antigen-binding fragments thereof, comprising a heavy chain complementarity determining region (CDR) having an amino acid sequence at least 90% identical to at least one of the amino acid sequences set forth as SEQ ID NOS: 4-6, 12-14, 17-19, 21-23, 25-27, 29-31, and 33-35. Disclosed herein, in certain embodiments, are monoclonal antibodies or an antigen-binding fragments thereof, comprising a heavy chain complementarity determining region (CDR) having an amino acid sequence at least 95% identical to at least one of the amino acid sequences set forth as SEQ ID NOS: 4-6, 12-14, 17-19, 21-23, 25-27, 29-31, and 33-35. Disclosed herein, in certain embodiments, are monoclonal antibodies or an antigen-binding fragments thereof, comprising a heavy chain complementarity determining region (CDR) having an amino acid sequence at least 99% identical to at least one of the amino acid sequences set forth as SEQ ID NOS: 4-6, 12-14, 17-19, 21-23, 25-27, 29-31, and 33-35. Disclosed herein, in certain embodiments, are monoclonal antibodies or an antigen-binding fragments thereof, comprising a heavy chain complementarity determining region (CDR) having an amino acid sequence 100% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment comprises a light chain complementarity determining region (CDR) having an amino acid sequence at least 80% identical to at least one of the amino acid sequences set forth as SEQ ID NOS: 1-3, and 9-11. In some instances, the monoclonal antibody or antigen-binding fragment comprises a light chain complementarity determining region (CDR) having an amino acid sequence at least 90% identical to at least one of the amino acid sequences set forth as SEQ ID NOS: 1-3, and 9-11. In some instances, the monoclonal antibody or antigen-binding fragment comprises a light chain complementarity determining region (CDR) having an amino acid sequence at least 95% identical to at least one of the amino acid sequences set forth as SEQ ID NOS: 1-3, and 9-11. In some instances, the monoclonal antibody or antigen-binding fragment comprises a light chain complementarity determining region (CDR) having an amino acid sequence at least 99% identical to at least one of the amino acid sequences set forth as SEQ ID NOS: 1-3, and 9-11. In some instances, the monoclonal antibody or antigen-binding fragment comprises a light chain complementarity determining region (CDR) having an amino acid sequence 100% identical to at least one of the amino acid sequences set forth as SEQ ID NOS: 1-3, and 9-11. In some instances, the monoclonal antibody or antigen-binding fragment thereof comprises a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35, a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, and a light chain complementarity determining region 3 (CDR3) having an amino acid sequence at least 80% identical to one of SEQ ID NO: 3, or SEQ ID NO: 11. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 17, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 19. In some instances, the monoclonal antibody or antigen-binding fragment thereof comprises at least one of a heavy chain complementarity determining region 1 (CDR1) having an amino acid sequence at least 80% identical to SEQ ID NO: 21, a heavy chain complementarity determining region 2 (CDR2) having an amino acid sequence at least 80% identical to SEQ ID NO: 22, and a heavy chain complementarity determining region 3 (CDR3) having an amino acid sequence at least 80% identical to SEQ ID NO: 23. In some instances, the monoclonal antibody or antigen-binding fragment thereof comprises at least one of a heavy chain complementarity determining region 1 (CDR1) having an amino acid sequence at least 80% identical to SEQ ID NO: 25, a heavy chain complementarity determining region 2 (CDR2) having an amino acid sequence at least 80% identical to SEQ ID NO: 26, and a heavy chain complementarity determining region 3 (CDR3) having an amino acid sequence at least 80% identical to SEQ ID NO: 27. In some instances, the monoclonal antibody or antigen-binding fragment thereof comprises at least one of a heavy chain complementarity determining region 1 (CDR1) having an amino acid sequence at least 80% identical to SEQ ID NO: 29, a heavy chain complementarity determining region 2 (CDR2) having an amino acid sequence at least 80% identical to SEQ ID NO: 30, and a heavy chain complementarity determining region 3 (CDR3) having an amino acid sequence at least 80% identical to SEQ ID NO: 31. In some instances, the monoclonal antibody or antigen-binding fragment thereof comprises at least one of a heavy chain complementarity determining region 1 (CDR1) having an amino acid sequence at least 80% identical to SEQ ID NO: 33, a heavy chain complementarity determining region 2 (CDR2) having an amino acid sequence at least 80% identical to SEQ ID NO: 34, and a heavy chain complementarity determining region 3 (CDR3) having an amino acid sequence at least 80% identical to SEQ ID NO: 35. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37; and a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to SEQ ID NO: 8 and a light chain variable domain (VL) comprising an amino acid sequence at least 80% identical to SEQ ID NO: 7. In some instances, the monoclonal antibody or antigen-binding fragment thereof comprises a heavy chain variable domain (VH) comprising an amino acid sequence at least 80% identical to SEQ ID NO: 16 and a light chain variable domain (VL) comprising an amino acid sequence at least 80% identical to SEQ ID NO: 15. In some instances, the monoclonal antibody or antigen-binding fragment thereof selectively binds to a complex comprising an HLA-E and a neoantigen. In some instances, the monoclonal antibody or antigen-binding fragment thereof does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL). In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL (SEQ ID NO: 38). In some instances, the monoclonal antibody or antigen-binding fragment thereof is a murine antibody, a chimeric antibody, a camelid antibody, a humanized antibody, or a human antibody. In some instances, the monoclonal antibody or antigen-binding fragment thereof is a TCR-like antibody. In some instances, the monoclonal antibody or antigen-binding fragment thereof is a multispecific antibody. In some instances, the monoclonal antibody or antigen-binding fragment thereof is a multifunctional antibody. In some instances, the monoclonal antibody or antigen-binding fragment thereof antibody further comprises a conjugated therapeutic moiety. In some instances, the selective binding of the antibody to the complex comprising the HLA-E and the neoantigen induces an immune response in a cell. In some instances, the immune response comprises activation of T cells. In some instances, the T cell is a CD8+ T cell. In some instances, the immune response comprises activation of cytotoxic T cells (CTLs). In some instances, the cell is a cancer cell.

Disclosed herein, in some embodiments, are pharmaceutical compositions comprising: a monoclonal antibody or an antigen-binding fragment thereof according to any of the disclosures herein; and a pharmaceutically acceptable carrier or excipient.

Disclosed herein, in some embodiments, are methods of treating cancer in an individual in need thereof, comprising administering to the individual an effective amount of a monoclonal antibody or an antigen-binding fragment thereof comprising a light chain complementarity determining region (CDR) having an amino acid sequence at least 80% identical to at least one of the amino acid sequences set forth as SEQ ID NOS: 1-3, or 9-11. Disclosed herein, in some embodiments, are methods of treating cancer in an individual in need thereof, comprising administering to the individual an effective amount of a monoclonal antibody or an antigen-binding fragment thereof comprising a heavy chain complementarity determining region (CDR) having an amino acid sequence at least 80% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises (a) a light chain complementarity determining region (CDR) having an amino acid sequence at least 80% identical to at least one of the amino acid sequences set forth as SEQ ID NOS: 1-3, or 9-11; and (b) a heavy chain complementarity determining region (CDR) having an amino acid sequence at least 80% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises (a) a light chain complementarity determining region (CDR) having an amino acid sequence at least 90% identical to at least one of the amino acid sequences set forth as SEQ ID NOS: 1-3, or 9-11; and (b) a heavy chain complementarity determining region (CDR) having an amino acid sequence at least 90% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises (a) a light chain complementarity determining region (CDR) having an amino acid sequence at least 95% identical to at least one of the amino acid sequences set forth as SEQ ID NOS: 1-3, or 9-11; and (b) a heavy chain complementarity determining region (CDR) having an amino acid sequence at least 95% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises (a) a light chain complementarity determining region (CDR) having an amino acid sequence at least 99% identical to at least one of the amino acid sequences set forth as SEQ ID NOS: 1-3, or 9-11; and (b) a heavy chain complementarity determining region (CDR) having an amino acid sequence at least 99% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises (a) a light chain complementarity determining region (CDR) having an amino acid sequence 100% identical to at least one of the amino acid sequences set forth as SEQ ID NOS: 1-3, or 9-11; and (b) a heavy chain complementarity determining region (CDR) having an amino acid sequence 100% identical to at least one of the amino acid sequences set forth as SEQ ID NOS: 4-6, 12-14, 17-19, 21-23, 25-27, 29-31, and 33-35. In some instances, the monoclonal antibody or antigen-binding fragment thereof comprises a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some instances, the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3. In some instances, the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11. In some instances, the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6. In some instances, the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14. In some instances, the monoclonal antibody or antigen-binding fragment thereof comprises at least one of a heavy chain complementarity determining region 1 (CDR1) having an amino acid sequence at least 80% identical to SEQ ID NO: 17, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 19. In some instances, the monoclonal antibody or antigen-binding fragment thereof comprises at least one of a heavy chain complementarity determining region 1 (CDR1) having an amino acid sequence at least 80% identical to SEQ ID NO: 21, a heavy chain complementarity determining region 2 (CDR2) having an amino acid sequence at least 80% identical to SEQ ID NO: 22, and a heavy chain complementarity determining region 3 (CDR3) having an amino acid sequence at least 80% identical to SEQ ID NO: 23. In some instances, the monoclonal antibody or antigen-binding fragment thereof comprises at least one of a heavy chain complementarity determining region 1 (CDR1) having an amino acid sequence at least 80% identical to SEQ ID NO: 25, a heavy chain complementarity determining region 2 (CDR2) having an amino acid sequence at least 80% identical to SEQ ID NO: 26, and a heavy chain complementarity determining region 3 (CDR3) having an amino acid sequence at least 80% identical to SEQ ID NO: 27. In some instances, the monoclonal antibody or antigen-binding fragment thereof comprises at least one of a heavy chain complementarity determining region 1 (CDR1) having an amino acid sequence at least 80% identical to SEQ ID NO: 29, a heavy chain complementarity determining region 2 (CDR2) having an amino acid sequence at least 80% identical to SEQ ID NO: 30, and a heavy chain complementarity determining region 3 (CDR3) having an amino acid sequence at least 80% identical to SEQ ID NO: 31. In some instances, the monoclonal antibody or antigen-binding fragment thereof comprises at least one of a heavy chain complementarity determining region 1 (CDR1) having an amino acid sequence at least 80% identical to SEQ ID NO: 33, a heavy chain complementarity determining region 2 (CDR2) having an amino acid sequence at least 80% identical to SEQ ID NO: 34, and a heavy chain complementarity determining region 3 (CDR3) having an amino acid sequence at least 80% identical to SEQ ID NO: 35. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15. In some instances, the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15; and a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some instances, the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8. In some instances, the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16. In some instances, the monoclonal antibody or antigen-binding fragment thereof selectively binds to a complex comprising an HLA-E and a neoantigen. In some instances, the monoclonal antibody or antigen-binding fragment thereof does not have a binding affinity to (i) the HLA-E alone; or (ii) the neoantigen alone. In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL). In some instances, the HLA-E is HLA-E*0101 or HLA-E*0103. In some instances, the antibody selectively binds to the complex comprising: (a) the HLA-E*0101 and the neoantigen; (b) the HLA-E*0103 and the neoantigen; or (c) the HLA-E*0101 and the neoantigen, and the HLA-E*0103 and the neoantigen. In some instances, the complex comprises the HLA-E and VMAPRTLFL (SEQ ID NO: 38). In some instances, the monoclonal antibody or antigen-binding fragment thereof is a murine antibody, a chimeric antibody, a camelid antibody, a humanized antibody, or a human antibody. In some instances, the monoclonal antibody or antigen-binding fragment thereof is a TCR-like antibody. In some instances, the monoclonal antibody or antigen-binding fragment thereof is a multispecific antibody. In some instances, the monoclonal antibody or antigen-binding fragment thereof is a multifunctional antibody. In some instances, the monoclonal antibody or antigen-binding fragment thereof antibody further comprises a conjugated therapeutic moiety. In some instances, the selective binding of the antibody to the complex comprising the HLA-E and the neoantigen induces an immune response in a cell. In some instances, the immune response comprises activation of T cells. In some instances, the T cell is a CD8+ T cell. In some instances, the immune response comprises activation of cytotoxic T cells (CTLs). In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount. In some instances, the cancer is breast cancer, kidney cancer, lung cancer, ovarian cancer, or colorectal cancer. In some instances, the cancer is a B-cell malignancy.

Disclosed herein, in some embodiments, are monoclonal antibodies or an antigen-binding fragments thereof according to any of the disclosures herein for use in treating cancer in an individual in need thereof. Disclosed herein, in some embodiments, are monoclonal antibodies or an antigen-binding fragments thereof according to any of the disclosures herein for use in preparation of a medicament for treating cancer in an individual in need thereof. In some instances, the cancer is breast cancer, kidney cancer, lung cancer, ovarian cancer, or colorectal cancer. In some instances, the cancer is a B-cell malignancy.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1B exemplify binding specificity and sensitivity of MAB-031 (anti-HLA-E/VMAPRTLFL) by ELISA. FIG. 1A shows monovalent binding of immobilized MAB-031 to decreasing concentrations (1 ug/ml to 0.0001 ug/ml) of soluble HLA-E/VMAPRTLFL complex. MAB-031 does not bind HLA-E complexes loaded with irrelevant peptides. FIG. 1B shows titration of soluble MAB-031 (IgG1 format) from 1.0 to 0.0001 ug/ml. Mab-031 binding to immobilized HLA-E/VMAPRTLFL complexes was observed at a concentration of 0.001 ug/ml. Binding of MAB-031 to HLA-E loaded with irrelevant peptides was not observed.

FIG. 2A-FIG. 2B exemplify binding specificity and sensitivity of MAB-036 (anti-HLA-E/VMAPRTLFL) by ELISA. FIG. 2A shows monovalent binding of immobilized MAB-036 to decreasing concentrations (1 ug/ml to 0.0001 ug/ml) of soluble HLA-E/VMAPRTLFL complex. MAB-036 does not bind HLA-E complexes loaded with irrelevant peptides. FIG. 2B shows titration of soluble MAB-036 (IgG1 format) from 1.0 to 0.0001 ug/ml. Mab-036 binding to immobilized HLA-E/VMAPRTLFL complexes was observed at concentrations ranging from 1 ug/ml to 0.001 ug/ml. Binding of MAB-036 to HLA-E loaded with irrelevant peptides was not observed.

FIG. 3A-FIG. 3C exemplify target specific recognition on tumor cells by MAB-031 and MAB-036. JEG-3 cells (positive for HLA-E and HLA-G) were stained with antibodies 3D12-APC (anti-HLA-E) FIG. 3A, MAB-031 (anti-HLA-E/VMAPRTLFL) FIG. 3B, and MAB-036 (anti-HLA-E/VMAPRTLFL) FIG. 3C. MOPC-21, a mouse IgG1 isotype was used as a control antibody for 3D12. A human IgG1 isotype antibody was used as a control for MAB-031 and MAB-036. Goat anti-human IgG-APC secondary conjugate was used to detect cell bound MAB-031 and MAB-036.

FIG. 4A-FIG. 4C exemplify target specific recognition on tumor cells by MAB-031 and MAB-036. JVM-2 cells (positive for HLA-E and HLA-G) were stained with antibodies 3D12-APC (anti-HLA-E) FIG. 4A, MAB-031 (anti-HLA-E/VMAPRTLFL) FIG. 4B, and MAB-036 (anti-HLA-E/VMAPRTLFL) FIG. 4C. MOPC-21, a mouse IgG1 isotype was used as a control antibody for 3D12. A human IgG1 isotype antibody was used as a control for MAB-031 and MAB-036. Goat anti-human IgG-APC secondary conjugate was used to detect cell bound MAB-031 and MAB-036.

FIG. 5A-FIG. 5C exemplify an absence of MAB-031 and MAB-036 binding to A549 cells (negative for HLA-E). A549 tumor cells were stained with antibodies 3D12-APC (anti-HLA-E) FIG. 5A, MAB-031 (anti-HLA-E/VMAPRTLFL) FIG. 5B and MAB-036 (anti-HLA-E/VMAPRTLFL) FIG. 5C. MOPC-21, a mouse IgG1 isotype was used as a control antibody for 3D12. A human IgG1 isotype antibody was used as a control for MAB-031 and MAB-036. Goat anti-human IgG-APC secondary conjugate was used to detect cell bound MAB-031 and MAB-036.

FIG. 6A-FIG. 6C exemplify an absence of MAB-031 and MAB-036 binding to EB-1 cells (positive for HLA-E). EB-1 tumor cells were stained with antibodies 3D12-APC (anti-HLA-E) FIG. 6A, MAB-031 (anti-HLA-E/VMAPRTLFL) FIG. 6B and MAB-036 (anti-HLA-E/VMAPRTLFL) FIG. 6C. MOPC-21, a mouse IgG1 isotype was used as a control antibody for 3D12. A human IgG1 isotype antibody was used as a control for MAB-031 and MAB-036. Goat anti-human IgG-APC secondary conjugate was used to detect cell bound MAB-031 and MAB-036.

### DETAILED DESCRIPTION

Disclosed herein, in certain embodiments, are antibodies that selectively bind to a complex comprising a non-classical HLA-I (e.g. HLA-E) and a neoantigen. Further disclosed herein, in certain embodiments, are methods of treating a cancer by administering an antibody that selectively binds to a complex comprising a non-classical HLA-I (e.g. HLA-E) and a neoantigen. In some embodiments, the antibodies that selectively bind to a complex comprising a non-classical HLA-I (e.g. HLA-E) and a neoantigen modulate immune response against cancer cells, thereby treating cancer.

Traditional approaches to the treatment of cancers have included surgery, radiation, chemotherapy and hormone therapy. However, such therapies have not proven effective by themselves. Development of alternate remedies for preventing and/or treating cancer is crucial. More recently immunotherapy and gene therapy approaches utilizing antibodies and T-lymphocytes have emerged as new and promising methods for treating cancer.

Major histocompatibility complex (MHC) molecules, designated human leukocyte antigen (HLA) in humans, play a critical role in the body's recognition of disease and the resulting immune response to cancer and invading antigens. The HLA gene family is divided into two subgroups namely HLA Class I (HLA-I) and HLA Class II (HLA-II), with HLA-I further divided into classical HLA-I and non-classical HLA-I. Each HLA molecule forms a complex with one peptide from within the cell. On cancer cells, some of the peptide/HLA complexes are uniquely presented which enables the immune system to recognize and kill these cells. Cells decorated with these unique peptide/HLA complexes are recognized and killed by the cytotoxic T cells (CTLs). Cancer cells show a downregulation in classical HLA-I expression but an upregulation in non-classical HLA-I expression (e.g. HLA-E). Thus, the upregulated uniquely presented non-classical HLA-I-peptide complexes on cancer cells are novel targets for developing innovative immunotherapies for treatment of cancer.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the term “MHC” refers to the Major Histocompability Complex, which is a set of gene loci specifying major histocompatibility antigens. The term “HLA” as used herein refer to Human Leukocyte Antigens, which are the histocompatibility antigens found in humans. As used herein, “HLA” is the human form of “MHC” and the terms are used interchangeably.

As used herein “antibody” refers to a glycoprotein which exhibits binding specificity to a specific antigen. Antibodies herein also include “antigen binding portion” or fragments of the antibody that are capable of binding to the antigen. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

As used herein, the term “selectively binds” in the context of any binding agent, e.g., an antibody, refers to a binding agent that binds specifically to an antigen or epitope, such as with a high affinity, and does not significantly bind other unrelated antigens or epitopes.

As used herein the term “neoantigen” or “neopeptide” are used interchangeably and refer to a peptide expressed by a diseased or stressed cell (e.g. cancer cell).

The terms “recipient”, “individual”, “subject”, “host”, and “patient”, are used interchangeably herein and in some cases, refer to any mammalian subject for whom diagnosis, treatment, or therapy is desired, particularly humans. None of these terms require the supervision of medical personnel.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with diseases (e.g. cancer). The term “therapeutic effect” refers to the reduction, elimination, or prevention of the disease, symptoms of the disease, or side effects of the disease in the subject.

A “therapeutically effective amount” in some cases means the amount that, when administered to a subject for treating a disease, is sufficient to effect treatment for that disease.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

### Major Histocompability Complex (MHC) or Human Leukocyte Antigens (HLA)

Major histocompatibility complexes (MHC), also termed Human Leukocyte Antigens (HLA) in humans are glycoproteins expressed on the surface of nucleated cells that act as proteomic scanning chips by providing insight into the status of cellular health. They continuously sample peptides from normal host cellular proteins, cancer cells, inflamed cells and bacterial, viral and parasite infected cells and present short peptides on the surface of cells for recognition by T lymphocytes. Presented peptides can also be derived from proteins that are out of frame or from sequences embedded in the introns, or from proteins whose translation is initiated at codons other than the conventional methionine codon, ATG.

There are two classes of MHCs in mice and humans, namely MHC I and MHC II. MHC I comprises classical and non-classical MHC I sub groups.

### Classical MHC I or HLA-I

Classical MHC I molecules include HLA-A, HLA-B and HLA-C in humans and H-2-K, H-2-D, H-2-B and H-2-L in mice. Classical MHC I molecules are highly polymorphic with more than 2,735 alleles of HLA-A, 3,455 alleles of HLA-B and 2,259 alleles of HLA-C. Classical MHC I is expressed on the surface of all nucleated cells and present peptides to CD8 T lymphocytes. 30% of the proteins in the cellular machinery are rapidly degraded and are primary substrates for classical MHC I antigen presentation.

For peptide to be presented by classical MHC I molecules, proteins are first processed through the conventional processing route (ubiquitin proteasome system) which begins with protein degradation in the proteasome and Transporter associated protein (TAP) dependent transport of peptides into the endoplasmic reticulum (ER) and ends with the loading of peptides into the HLA peptide binding pocket. The proteins that contribute to the conventional processing route are collectively known as antigen processing machinery (APM) and include the proteasome, TAP complex, tapasin, endoplasmic reticulum amino peptidase (ERAAP), binding immunoglobulin protein (BiP), clanexin and calreticulin. Cells lacking either proteasome subunits, TAP1/2, ErP57 or calreticulin have reduced numbers of classical MHC I molecules on their surface.

### Non-Classical MHC I or HLA-I

Non-classical MHC I molecules include HLA-E, HLA-F and HLA-G, and have limited polymorphisms. They play a role in regulating innate and adaptive immune responses. Non-classical MHC I molecules present peptides generated by both the conventional processing route and the alternative processing route in health and disease states, and represent a novel set of markers for targeting in disease states (e.g. cancer).

### HLA-E

The non-classical MHC class I molecule, HLA-E is non-polymorphic. In nature, 13 HLA-E alleles have been identified with only two functional variants, namely HLAE* 0101 and HLA-E*0103. The difference between HLA-E*0101 (HLA-E^{107R}) and *0103 (HLA-E^{107G}) is a single amino acid difference at position 107 which is outside the peptide binding pocket. Similar to the classical MHC I molecules, HLA-E is expressed in all cells with a nucleus, however at usually lower levels. HLA-E molecule expression in cells and tissues is generally increased during stress and disease.

In healthy cells, HLA-E presents peptides derived from classical MHC molecules and the non-classical HLA-G molecule to either inhibit or stimulate the activity of NK cells and a subset of CD8 T cells through engaging the receptor CD94/NKG2. Depending on the particular peptide presented by HLA-E, the HLA-E complex engages either CD94/NKG2A or CD94/NKG2C to inhibit or activate NK cells and a subset of CD8 T cells, respectively.

Another signal peptide that has characteristics in common with signal peptides generated from classical HLA-I molecules is the signal peptide generated from non-classical HLA-G. HLA-G expression under normal physiologic conditions is tightly regulated, with limited expression found in relatively few tissues and cells in the body. HLA-G plays a key role as an immune tolerant molecule and its expression is observed in cancer tissue/cells. Moreover, the signal peptide from HLA-G is processed by the conventional antigen processing pathway and delivered to the endoplasmic reticulum by the peptide transporter TAP. In some instances, the signal peptide is VMAPRTLFL (SEQ ID NO: 38).

HLA-E Expression and Peptide Presentation in Cancer Cells

Cells deficient in one or more components of the APM load peptides into MHC class I molecules via alternative processing routes which are independent of the APM-dependent conventional processing route. APM-deficient cells not only have reduced numbers of classical MHC I molecules on their surface, but also show an increase in the cell surface density of HLA-E molecules as well as an increase in the repertoire of peptides presented. The alternative processing routes are constitutively turned on and produce peptides in both healthy and diseased cells. These peptides, however, are not presented by healthy cells; instead they are only presented in diseased or stressed cells. As such, the different peptide repertoires generated by APM-defective cells, also known as “T-cell epitopes associated with impaired peptide processing” (TEIPP), represent novel targets unique to cancer cells, and represent ideal targets for therapeutic development in the treatment of cancer.

### MHC II or HLA-II

MHC II molecules in humans include HLA-DM, HLA-DO, HLA-DP, HLA-DQ and HLA-DR and include H-2 I-A and H-2 I-E in mice. MHC II expression is more restricted to B cells, dendritic cells, macrophages, activated T cells and thymic epithelial cells and MHC II molecules present peptides to CD4 lymphocytes.

Antibodies that Target a Complex Comprising a Non-Classical HLA-I (e.g. HLA-E) and a Neoantigen

Disclosed herein, in certain embodiments, are antibodies that target a complex comprising a non-classical HLA-I (e.g. HLA-E) and a neoantigen. In some embodiments, the antibodies comprise at least one heavy chain comprising a heavy chain variable domain (VH) and at least one light chain comprising a light chain variable domain (VL). Each VH and VL comprises three complementarity determining regions (CDR). The amino acid sequences of the VH and VL and the CDRs determine the antigen binding specificity and antigen binding strength of the antibody. The amino acid sequences of the VH and VL and the CDRs are summarized in Table 1.

TABLE 1
Antibodies that bind a complex comprising HLA-E and
VMAPRTLFL (SEQ ID NO: 38)

| | |
|---|---|
| | SEQ ID |
| SEQUENCE | NO: |

Human monoclonal antibody sequences

| | | |
|---|---|---|
| Mab-036 Light Chain | QSISSY | 1 |
| CDR1 | | |
| Mab-036 Light Chain | NAA | 2 |
| CDR2 | | |
| Mab-036 Light Chain | QQAATYPAT | 3 |
| CDR3 | | |
| Mab-036 Heavy Chain | GFTFSSYA | 4 |
| CDR1 | | |
| Mab-036 Heavy Chain | IAYGGGAT | 5 |
| CDR2 | | |
| Mab-036 Heavy Chain | AKGLSNFDY | 6 |
| CDR3 | | |
| Mab-036 Light Chain | DIQMTQSPSSLSASVGDRVTITCRASQSISSYLNWYQQKPG | 7 |
| Variable domain | KAPKLLIYNAAFLYSGVPSRFSGSRSGTDFTLTISSLQPEDFA | |
| | TYYCQQAATYPATFGQGTKVEIK | |
| Mab-036 Heavy Chain | EVQLVESGGGLVQPGGSLRLSCAASGFTFSSYAIHWVRQAP | 8 |
| Variable domain | GKGLEWVARIAYGGGATRYADSVKGRFTISADTSKNTAYL | |
| | QMNSLRAEDTAVYYCAKGLSNFDYWGQGTLVTVSS | |

Mouse monoclonal antibody sequences

| | | |
|---|---|---|
| Mab-031 Light Chain | SSVSY | 9 |
| CDR1 | | |
| Mab-031 Light Chain | STS | 10 |
| CDR2 | | |
| Mab-031 Light Chain | QQRSSYPPT | 11 |
| CDR3 | | |
| Mab-031 Heavy Chain | GYTFTDYV | 12 |
| CDR1 | | |
| Mab-031 Heavy Chain | IYPGNNTT | 13 |
| CDR2 | | |
| Mab-031 Heavy Chain | ARESLGIYYGSTYKGLYAMDY | 14 |
| CDR3 | | |
| Mab-031 Light Chain | QIVLTQSPAIMSASPGEKVTITCSASSSVSYMHWFQQKPGTS | 15 |
| Variable domain | PKLWIYSTSNLASGVPARFSGSGSGTSYSLTISRMEAEDAAT | |
| | YYCQQRSSYPPTFGAGTKLELKRT | |
| Mab-031 Heavy Chain | QVQLQQSGPELVKPGASVKMSCKASGYTFTDYVISWVKQR | 16 |
| Variable domain | TGQGLEWIGEIYPGNNTTYYNDNFRGKATLTVDKSSSTAY | |
| | MQLSSLTSEDSAVYYCARESLGIYYGSTYKGLYAMDYWG | |
| | QGTTLTVSS | |

Llama monoclonal antibody sequences

| | | |
|---|---|---|
| Mab-024 Heavy Chain | GIIFSDYR | 17 |
| CDR1 | | |
| Mab-024 Heavy Chain | ISTGGSI | 18 |
| CDR2 | | |
| Mab-024 Heavy Chain | NAGLR | 19 |
| CDR3 | | |
| Mab-024 Heavy Chain | MAQVQLQESGGGLVQAGGSLRLTCVASGIIFSDYRVAWYR | 20 |
| Variable domain | QAPGKQRELVARISTGGSILYADSVKGRFTISRDNAKNTVN | |
| | LQMNSLKPEDTAVYYCNAGLRWGQGTQVTVSS | |
| Mab-025 Heavy Chain | GRTFSRAT | 21 |
| CDR1 | | |
| Mab-025 Heavy Chain | IRWSTEST | 22 |
| CDR2 | | |
| Mab-025 Heavy Chain | ATDLSTYYGAIDTGADDYDY | 23 |
| CDR3 | | |
| Mab-025 Heavy Chain | MAQVTLKESGGGLVQAGGSLRLSCAVSGRTFSRATMAWF | 24 |
| Variable domain | RQAPGKEREFVAGIRWSTESTYYADSVKGRFTISRDRAKNT | |
| | VYLDMNSLKPEDTAAYYCATDLSTYYGAIDTGADDYDYW | |
| | GQGTQVTVSS | |
| Mab-026 Heavy Chain | GRISTTYA | 25 |
| CDR1 | | |
| Mab-026 Heavy Chain | IYWSGGMT | 26 |
| CDR2 | | |
| Mab-026 Heavy Chain | AADPRAAYYYGTSDYTLPGRYNN | 27 |
| CDR3 | | |
| Mab-026 Heavy Chain | MAQVQLVQSGGGLVQPGGSLRLSCAASGRISTTYAMAWF | 28 |
| Variable domain | RQAPGKEREFVAAIYWSGGMTKYADSVKGRSTISRDNAKN | |
| | TVLLQMNSLKSGDTAVYYCAADPRAAYYYGTSDYTLPGR | |
| | YNNWGQGTQVTVSS | |
| Mab-027 Heavy Chain | GSISSFEQ | 29 |
| CDR1 | | |
| Mab-027 Heavy Chain | FRSDGST | 30 |
| CDR2 | | |
| Mab-027 Heavy Chain | NTYPVVLYN | 31 |
| CDR3 | | |
| Mab-027 Heavy Chain | MAQVTLKESGGGLVQPGGSLRLSCAASGSISSFEQMAWYR | 32 |
| Variable domain | QAPGKQRALVARFRSDGSTKYTDSVKDRFTISRDNAKSTV | |
| | YLQMNSLKPEDTAVYYCNTYPVVLYNWGQGTQVTVSS | |
| Mab-028 Heavy Chain | GRTFSHAT | 33 |
| CDR1 | | |
| Mab-028 Heavy Chain | ITWSTEST | 34 |
| CDR2 | | |
| Mab-028 Heavy Chain | AADLSTYYGAIDTGADDYDY | 35 |
| CDR3 | | |
| Mab-028 Heavy Chain | MAQVTLQESGGGLVQAGGSLRLSCAASGRTFSHATMAWF | 36 |
| Variable domain | RQAPGKEREFVARITWSTESTYYEYSVKGRFTISKDRAKNT | |
| | VYLDMNRLKPEDTAAYYCAADLSTYYGAIDTGADDYDY | |
| | WGQGTQVTVSS | |
| Mab-029 Heavy Chain | GRTFSRAT | 21 |
| CDR1 | | |
| Mab-029 Heavy Chain | IRWSTEST | 22 |
| CDR2 | | |
| Mab-029 Heavy Chain | ATDLSTYYGAIDTGADDYDY | 23 |
| CDR3 | | |
| Mab-029 Heavy Chain | MAQVQLQESGGGLVQAGGSLRLSCAVSGRTFSRATMAWF | 37 |
| Variable domain | RQAPGKEREFVAGIRWSTESTYYADSVKGRFTISRDRAKNT | |
| | VYLDMNSLKPEDTAAYYCATDLSTYYGAIDTGADDYDYW | |
| | GQGTQVTVSS | |

In some embodiments, the antibodies selectively bind to a complex comprising a non-classical HLA-I (e.g. HLA-E) and a neoantigen. In some instances, the antibody does not have a binding affinity to the non-classical HLA-I alone. In some instances, the antibody does not have a binding affinity to the neoantigen alone. In some instances, the antibody does not have a binding affinity to a complex comprising the non-classical HLA-I and a non-relevant neoantigen.

In some instances, the neoantigen is expressed by an antigen processing machinery (APM)-proficient cell. In some instances, the neoantigen is expressed by a TA-P1/2-proficient cell. In some instances, the neoantigen is expressed by an antigen processing machinery (APM)-deficient cell. In some instances, the neoantigen comprises, consisting essentially of, or consisting of a sequence according to SEQ TD NO: 38 (VMAPRTLFL).

In some instances, the non-classical HLA-I is HLA-E, HLA-F, HLA-G, or HLA-H. In some instances, the non-classical HLA-I is HLA-E. In some instances, the HLA-E is HLA-E*0101. In some instances, the HLA-E is HLA-E*0103.

In some instances, the antibody selectively binds to the complex comprising the HLA-E and the neoantigen. In some instances, the antibody selectively binds to the complex comprising the HLA-E*0101 and the neoantigen. In some instances, the antibody selectively binds to the complex comprising the HLA-E*0103 and the neoantigen. In some instances, the antibody selectively binds to the complex comprising the HLA-E*0101 and the neoantigen, and to the complex of the HLA-E*0103 and the neoantigen. In some instances, the complex comprises the HLA-E and VMAPRTLFL (SEQ ID NO: 38).

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I (e.g. HLA-E) and the neoantigen induces an immune response. In some instances, the immune response comprises activation of T cells. In some instances, the T cell is a CD8+ T cell. In some instances, the immune response comprises activation of cytotoxic T cells (CTLs). In some instances, the cell is a cancer cell.

### Antibody Variable Domain (VL and VH)

Disclosed herein are antibodies that selectively bind to a complex comprising an HLA-E and VMAPRTLFL (SEQ ID NO: 38) having a light chain comprising a light chain variable domain (VL). In some embodiments, antibodies comprise a light chain variable domain (VL) having an amino acid sequence at least about 70% identical to an amino acid sequence set forth as SEQ ID NO: 7, or SEQ ID NO: 15. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, or SEQ ID NO: 15. In some embodiments, the VL has an amino acid sequence 100% identical to an amino acid sequence set forth as SEQ ID NO: 7, or SEQ ID NO: 15.

Further disclosed herein are antibodies that selectively bind to a complex comprising an HLA-E and VMAPRTLFL (SEQ ID NO: 38) having a heavy chain comprising a heavy chain variable domain (VH). In some embodiments, antibodies comprise a heavy chain variable domain (VH) having an amino acid sequence at least about 70% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments, the VH has an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

Also disclosed herein are antibodies that selectively bind to a complex comprising an HLA-E and VMAPRTLFL (SEQ ID NO: 38) comprising a light chain variable domain (VL) and a heavy chain variable domain (VH). In some embodiments, antibodies comprise a light chain variable domain (VL) having an amino acid sequence at least about 70% identical to an amino acid sequence set forth as SEQ ID NO: 7, or SEQ ID NO: 15 and a heavy chain variable domain (VH) having an amino acid sequence at least about 70% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, or SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments, the VL has an amino acid sequence 100% identical to an amino acid sequence set forth as SEQ ID NO: 7, or SEQ ID NO: 15 and the VH has an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

In some embodiments, antibodies that selectively bind to a complex comprising an HLA-E and VMAPRTLFL (SEQ ID NO: 38) comprise a light chain variable domain (VL) having an amino acid sequence at least about 70% identical to an amino acid sequence set forth as SEQ ID NO: 7 and a heavy chain variable domain (VH) having an amino acid sequence at least about 70% identical to an amino acid sequence set forth as SEQ ID NO: 8. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VL has an amino acid sequence 100% identical to an amino acid sequence set forth as SEQ ID NO: 7 and the VH has an amino acid sequence 100% identical to an amino acid sequence set forth as SEQ ID NO: 8.

In some embodiments, antibodies that selectively bind to a complex comprising an HLA-E and VMAPRTLFL (SEQ ID NO: 38) comprise a light chain variable domain (VL) having an amino acid sequence at least about 70% identical to an amino acid sequence set forth as SEQ ID NO: 15 and a heavy chain variable domain (VH) having an amino acid sequence at least about 70% identical to an amino acid sequence set forth as SEQ ID NO: 16. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 16. In some embodiments, the VL has an amino acid sequence 100% identical to an amino acid sequence set forth as SEQ ID NO: 15 and the VH has an amino acid sequence 100% identical to an amino acid sequence set forth as SEQ ID NO: 16.

### Antibody Complementarity Determining Regions (CDR)

Disclosed herein are antibodies that selectively bind to a complex comprising an HLA-E and VMAPRTLFL (SEQ ID NO: 38) having a light chain comprising a light chain complementarity determining region (CDR). In some embodiments, antibodies comprise a light chain CDR sequence having an amino acid sequence at least about 70% identical to at least one of the amino acid sequences set forth as SEQ ID NOS: 1-3, or 9-11. In some embodiments, antibodies comprise a light chain CDR sequence having an amino acid sequence at least about 75%, 80%, 81%, 82%, 83%, 84%, 85%, 86%, 87%, 88%, 89%, 90%9, 91%, 92%, 93%, 94%, 95%, 96%, 97%, 98%, or 99% identical to at least one of the amino acid sequences set forth as SEQ ID NOS: 1-3, or 9-11. In some embodiments, antibodies comprise a light chain CDR sequence having an amino acid sequence 100% identical to at least one of the amino acid sequences set forth as SEQ ID NOS: 1-3, or 9-11.

Further disclosed herein are antibodies that selectively bind to a complex comprising an HLA-E and VMAPRTLFL (SEQ ID NO: 38) having a heavy chain comprising a heavy chain complementarity determining region (CDR). In some embodiments, antibodies comprise a heavy chain CDR sequence having an amino acid sequence at least about 70% identical to at least one of the amino acid sequences set forth as SEQ ID NOS: 4-6, 12-14, 17-19, 21-23, 25-27, 29-31, and 33-35. In some embodiments, antibodies comprise a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, 17-19, 21-23, 25-27, 29-31, and 33-35. In some embodiments, antibodies comprise a heavy chain CDR sequence having an amino acid sequence 100% identical to at least one of the amino acid sequences set forth as SEQ ID NOS: 4-6, 12-14, 17-19, 21-23, 25-27, 29-31, and 33-35.

Also disclosed herein are antibodies that selectively bind to a complex comprising an HLA-E and VMAPRTLFL (SEQ ID NO: 38) comprise a light chain complementarity determining region (CDR) and a heavy chain complementarity determining region (CDR). In some embodiments, antibodies comprise a light chain CDR sequence having an amino acid sequence at least about 70% identical to at least one of the amino acid sequences set forth as SEQ ID NOS: 1-3, or 9-11 and a heavy chain CDR sequence having an amino acid sequence at least about 70% identical to at least one of the amino acid sequences set forth as SEQ ID NOS: 4-6, 12-14, 17-19, 21-23, 25-27, 29-31, and 33-35. In some embodiments, antibodies comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, or 9-11 and a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, 17-19, 21-23, 25-27, 29-31, and 33-35. In some embodiments, antibodies comprise a light chain CDR sequence having an amino acid sequence 100% identical to at least one of the amino acid sequences set forth as SEQ ID NOS: 1-3, or 9-11 and a heavy chain CDR sequence having an amino acid sequence at least about 100% identical to at least one of the amino acid sequences set forth as SEQ ID NOS: 4-6, 12-14, 17-19, 21-23, 25-27, 29-31, and 33-35.

In some embodiments, antibodies that selectively bind to a complex comprising an HLA-E and VMAPRTLFL (SEQ ID NO: 38) comprise a light chain complementarity determining region 1 (CDR1) having an amino acid sequence at least about 70% identical to one of SEQ ID NO: 1, or SEQ ID NO: 9, a light chain complementarity determining region 2 (CDR2) having an amino acid sequence at least about 70% identical to one of SEQ ID NO: 2, or SEQ ID NO: 10, a light chain complementarity determining region 3 (CDR3) having an amino acid sequence at least about 70% identical to one of SEQ ID NO: 3, or SEQ ID NO: 11, a heavy chain complementarity determining region 1 (CDR1) having an amino acid sequence at least about 70% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least about 70% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least about 70% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some embodiments, antibodies comprise a light chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 1, or SEQ ID NO: 9, a light chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 2, or SEQ ID NO: 10, a light chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 3, or SEQ ID NO: 11, a heavy chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some embodiments, antibodies comprise a light chain complementarity determining region 1 (CDR1) having an amino acid sequence 100% identical to one of SEQ ID NO: 1, or SEQ ID NO: 9, a light chain complementarity determining region 2 (CDR2) having an amino acid sequence 100% identical to one of SEQ ID NO: 2, or SEQ ID NO: 10, a light chain complementarity determining region 3 (CDR3) having an amino acid sequence 100% identical to one of SEQ ID NO: 3, or SEQ ID NO: 11, a heavy chain complementarity determining region 1 (CDR1) having an amino acid sequence 100% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence 100% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence 100% identical to one SEQ ID NO: 6, SEQ ID NO: 14, SEQ ID NO: 19, SEQ ID NO: 23, SEQ ID NO: 27, SEQ ID NO: 31, or SEQ ID NO: 35.

In some embodiments, antibodies selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, and a light chain CDR3 having an amino acid sequence at least about 70% identical to an amino acid sequence set forth as SEQ ID NO: 11. In some embodiments, antibodies comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11. In some embodiments, antibodies comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, and a light chain CDR3 having an amino acid sequence 100% identical to an amino acid sequence set forth as SEQ ID NO: 11.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, a heavy chain CDR3 having an amino acid sequence at least about 70% identical to an amino acid sequence set forth as SEQ ID NO: 6. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, a heavy chain CDR3 having an amino acid sequence 100% identical to an amino acid sequence set forth as SEQ ID NO: 6.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, a heavy chain CDR3 having an amino acid sequence at least about 70% identical to an amino acid sequence set forth as SEQ ID NO: 14. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies comprise at least one of a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, a heavy chain CDR3 having an amino acid sequence 100% identical to an amino acid sequence set forth as SEQ ID NO: 14.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 17, a heavy chain CDR2 having an amino acid sequence at least about 70% identical to an amino acid sequence set forth as SEQ ID NO: 18, a heavy chain CDR3 having an amino acid sequence at least about 70% identical to an amino acid sequence set forth as SEQ ID NO: 19. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 19. In some embodiments, antibodies comprise at least one of a heavy chain CDR1 having an amino acid sequence 100% identical to an amino acid sequence set forth as SEQ ID NO: 17, a heavy chain CDR2 having an amino acid sequence 100% identical to an amino acid sequence set forth as SEQ ID NO: 18, a heavy chain CDR3 having an amino acid sequence 100% identical to an amino acid sequence set forth as SEQ ID NO: 19.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 21, a heavy chain CDR2 having an amino acid sequence at least about 70% identical to an amino acid sequence set forth as SEQ ID NO: 22, a heavy chain CDR3 having an amino acid sequence at least about 70% identical to an amino acid sequence set forth as SEQ ID NO: 23. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23. In some embodiments, antibodies comprise at least one of a heavy chain CDR1 having an amino acid sequence 100% identical to an amino acid sequence set forth as SEQ ID NO: 21, a heavy chain CDR2 having an amino acid sequence 100% identical to an amino acid sequence set forth as SEQ ID NO: 22, a heavy chain CDR3 having an amino acid sequence 100% identical to an amino acid sequence set forth as SEQ ID NO: 23.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 25, a heavy chain CDR2 having an amino acid sequence at least about 70% identical to an amino acid sequence set forth as SEQ ID NO: 26, a heavy chain CDR3 having an amino acid sequence at least about 70% identical to an amino acid sequence set forth as SEQ ID NO: 27. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25, a heavy chain CDR2 having an amino acid sequence at least about 75%, 80%, 81%, 82%, 83%, 84%, 85%, 86%, 87%, 88%, 89%, 90%9, 91%, 92%, 93%, 94%, 95%, 96%, 97%, 98%, or 99% identical to an amino acid sequence set forth as SEQ ID NO: 26, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27. In some embodiments, antibodies comprise at least one of a heavy chain CDR1 having an amino acid sequence 100% identical to an amino acid sequence set forth as SEQ ID NO: 25, a heavy chain CDR2 having an amino acid sequence 100% identical to an amino acid sequence set forth as SEQ ID NO: 26, a heavy chain CDR3 having an amino acid sequence 100% identical to an amino acid sequence set forth as SEQ ID NO: 27.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 29, a heavy chain CDR2 having an amino acid sequence at least about 70% identical to an amino acid sequence set forth as SEQ ID NO: 30, a heavy chain CDR3 having an amino acid sequence at least about 70% identical to an amino acid sequence set forth as SEQ ID NO: 31. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, antibodies comprise at least one of a heavy chain CDR1 having an amino acid sequence 100% identical to an amino acid sequence set forth as SEQ ID NO: 29, a heavy chain CDR2 having an amino acid sequence 100% identical to an amino acid sequence set forth as SEQ ID NO: 30, a heavy chain CDR3 having an amino acid sequence 100% identical to an amino acid sequence set forth as SEQ ID NO: 31.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 33, a heavy chain CDR2 having an amino acid sequence at least about 70% identical to an amino acid sequence set forth as SEQ ID NO: 34, a heavy chain CDR3 having an amino acid sequence at least about 70% identical to an amino acid sequence set forth as SEQ ID NO: 35. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, antibodies comprise at least one of a heavy chain CDR1 having an amino acid sequence 100% identical to an amino acid sequence set forth as SEQ ID NO: 33, a heavy chain CDR2 having an amino acid sequence 100% identical to an amino acid sequence set forth as SEQ ID NO: 34, a heavy chain CDR3 having an amino acid sequence 100% identical to an amino acid sequence set forth as SEQ ID NO: 35.

In some embodiments, antibodies that selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, and a heavy chain CDR3 having an amino acid sequence at least about 70% identical to an amino acid sequence set forth as SEQ ID NO: 6. In some embodiments, antibodies comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, and a heavy chain CDR3 having an amino acid sequence 100% identical to an amino acid sequence set forth as SEQ ID NO: 6.

In some embodiments, antibodies that selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, a light chain CDR3 having an amino acid sequence at least about 70% identical to an amino acid sequence set forth as SEQ ID NO: 11, a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, and a heavy chain CDR3 having an amino acid sequence at least about 70% identical to an amino acid sequence set forth as SEQ ID NO: 14. In some embodiments, antibodies comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, a light chain CDR3 having an amino acid sequence 100% identical to an amino acid sequence set forth as SEQ ID NO: 11, a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, and a heavy chain CDR3 having an amino acid sequence 100% identical to an amino acid sequence set forth as SEQ ID NO: 14.

### Methods of Treatment

Provided herein are methods of treating cancer in an individual in need thereof comprising administration of antibodies that selectively bind to a complex comprising a non-classical HLA-I (e.g. HLA-E) and a neoantigen (e.g. VMAPRTLFL (SEQ ID NO: 38)) as disclosed herein.

Disclosed herein are antibodies that selectively bind to a complex comprising an HLA-E and VMAPRTLFL (SEQ ID NO: 38) having a light chain comprising a light chain complementarity determining region (CDR). In some embodiments, antibodies comprise a light chain CDR sequence having an amino acid sequence at least about 70% identical to at least one of the amino acid sequences set forth as SEQ ID NOS: 1-3, or 9-11. In some embodiments, antibodies comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, or 9-11. In some embodiments, antibodies comprise a light chain CDR sequence having an amino acid sequence 100% identical to at least one of the amino acid sequences set forth as SEQ ID NOS: 1-3, or 9-11.

Further disclosed herein are antibodies that selectively bind to a complex comprising an HLA-E and VMAPRTLFL (SEQ ID NO: 38) having a heavy chain comprising a heavy chain complementarity determining region (CDR). In some embodiments, antibodies comprise a heavy chain CDR sequence having an amino acid sequence at least about 70% identical to at least one of the amino acid sequences set forth as SEQ ID NOS: 4-6, 12-14, 17-19, 21-23, 25-27, 29-31, and 33-35. In some embodiments, antibodies comprise a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, 17-19, 21-23, 25-27, 29-31, and 33-35. In some embodiments, antibodies comprise a heavy chain CDR sequence having an amino acid sequence 100% identical to at least one of the amino acid sequences set forth as SEQ ID NOS: 4-6, 12-14, 17-19, 21-23, 25-27, 29-31, and 33-35.

Also disclosed herein are antibodies that selectively bind to a complex comprising an HLA-E and VMAPRTLFL (SEQ ID NO: 38) comprise a light chain complementarity determining region (CDR) and a heavy chain complementarity determining region (CDR). In some embodiments, antibodies comprise a light chain CDR sequence having an amino acid sequence at least about 70% identical to at least one of the amino acid sequences set forth as SEQ ID NOS: 1-3, or 9-11 and a heavy chain CDR sequence having an amino acid sequence at least about 70% identical to at least one of the amino acid sequences set forth as SEQ ID NOS: 4-6, 12-14, 17-19, 21-23, 25-27, 29-31, and 33-35. In some embodiments, antibodies comprise a light chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 1-3, or 9-11 and a heavy chain CDR sequence having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to at least one of the amino acid sequences set forth as SEQ ID NOS: 4-6, 12-14, 17-19, 21-23, 25-27, 29-31, and 33-35. In some embodiments, antibodies comprise a light chain CDR sequence having an amino acid sequence 100% identical to at least one of the amino acid sequences set forth as SEQ ID NOS: 1-3, or 9-11 and a heavy chain CDR sequence having an amino acid sequence at least about 100% identical to at least one of the amino acid sequences set forth as SEQ ID NOS: 4-6, 12-14, 17-19, 21-23, 25-27, 29-31, and 33-35.

In some embodiments, antibodies that selectively bind to a complex comprising an HLA-E and VMAPRTLFL (SEQ ID NO: 38) comprise a light chain complementarity determining region 1 (CDR1) having an amino acid sequence at least about 70% identical to one of SEQ ID NO: 1, or SEQ ID NO: 9, a light chain complementarity determining region 2 (CDR2) having an amino acid sequence at least about 70% identical to one of SEQ ID NO: 2, or SEQ ID NO: 10, a light chain complementarity determining region 3 (CDR3) having an amino acid sequence at least about 70% identical to one of SEQ ID NO: 3, or SEQ ID NO: 11, a heavy chain complementarity determining region 1 (CDR1) having an amino acid sequence at least about 70% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least about 70% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least about 70% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some embodiments, antibodies comprise a light chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 1, or SEQ ID NO: 9, a light chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 2, or SEQ ID NO: 10, a light chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 3, or SEQ ID NO: 11, a heavy chain complementarity determining region 1 (CDR1) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35. In some embodiments, antibodies comprise a light chain complementarity determining region 1 (CDR1) having an amino acid sequence 100% identical to one of SEQ ID NO: 1, or SEQ ID NO: 9, a light chain complementarity determining region 2 (CDR2) having an amino acid sequence 100% identical to one of SEQ ID NO: 2, or SEQ ID NO: 10, a light chain complementarity determining region 3 (CDR3) having an amino acid sequence 100% identical to one of SEQ ID NO: 3, or SEQ ID NO: 11, a heavy chain complementarity determining region 1 (CDR1) having an amino acid sequence 100% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence 100% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence 100% identical to one SEQ ID NO: 6, SEQ ID NO: 14, SEQ ID NO: 19, SEQ ID NO: 23, SEQ ID NO: 27, SEQ ID NO: 31, or SEQ ID NO: 35.

In some embodiments, antibodies selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, and a light chain CDR3 having an amino acid sequence at least about 70% identical to an amino acid sequence set forth as SEQ ID NO: 3. In some embodiments, antibodies comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3. In some embodiments, antibodies comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, and a light chain CDR3 having an amino acid sequence 100% identical to an amino acid sequence set forth as SEQ ID NO: 3.

In some embodiments, antibodies selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, and a light chain CDR3 having an amino acid sequence at least about 70% identical to an amino acid sequence set forth as SEQ ID NO: 11. In some embodiments, antibodies comprise at least one of a light chain a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, and a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11. In some embodiments, antibodies comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, and a light chain CDR3 having an amino acid sequence 100% identical to an amino acid sequence set forth as SEQ ID NO: 11.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, a heavy chain CDR3 having an amino acid sequence at least about 70% identical to an amino acid sequence set forth as SEQ ID NO: 6. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies comprise at least one of a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, a heavy chain CDR3 having an amino acid sequence 100% identical to an amino acid sequence set forth as SEQ ID NO: 6.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, a heavy chain CDR3 having an amino acid sequence at least about 70% identical to an amino acid sequence set forth as SEQ ID NO: 14. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies comprise at least one of a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, a heavy chain CDR3 having an amino acid sequence 100% identical to an amino acid sequence set forth as SEQ ID NO: 14.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 17, a heavy chain CDR2 having an amino acid sequence at least about 70% identical to an amino acid sequence set forth as SEQ ID NO: 18, a heavy chain CDR3 having an amino acid sequence at least about 70% identical to an amino acid sequence set forth as SEQ ID NO: 19. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 17, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 18, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 19. In some embodiments, antibodies comprise at least one of a heavy chain CDR1 having an amino acid sequence 100% identical to an amino acid sequence set forth as SEQ ID NO: 17, a heavy chain CDR2 having an amino acid sequence 100% identical to an amino acid sequence set forth as SEQ ID NO: 18, a heavy chain CDR3 having an amino acid sequence 100% identical to an amino acid sequence set forth as SEQ ID NO: 19.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 21, a heavy chain CDR2 having an amino acid sequence at least about 70% identical to an amino acid sequence set forth as SEQ ID NO: 22, a heavy chain CDR3 having an amino acid sequence at least about 70% identical to an amino acid sequence set forth as SEQ ID NO: 23. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 21, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 22, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 23. In some embodiments, antibodies comprise at least one of a heavy chain CDR1 having an amino acid sequence 100% identical to an amino acid sequence set forth as SEQ ID NO: 21, a heavy chain CDR2 having an amino acid sequence 100% identical to an amino acid sequence set forth as SEQ ID NO: 22, a heavy chain CDR3 having an amino acid sequence 100% identical to an amino acid sequence set forth as SEQ ID NO: 23.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 25, a heavy chain CDR2 having an amino acid sequence at least about 70% identical to an amino acid sequence set forth as SEQ ID NO: 26, a heavy chain CDR3 having an amino acid sequence at least about 70% identical to an amino acid sequence set forth as SEQ ID NO: 27. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 25, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 26, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 27. In some embodiments, antibodies comprise at least one of a heavy chain CDR1 having an amino acid sequence 100% identical to an amino acid sequence set forth as SEQ ID NO: 25, a heavy chain CDR2 having an amino acid sequence 100% identical to an amino acid sequence set forth as SEQ ID NO: 26, a heavy chain CDR3 having an amino acid sequence 100% identical to an amino acid sequence set forth as SEQ ID NO: 27.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 29, a heavy chain CDR2 having an amino acid sequence at least about 70% identical to an amino acid sequence set forth as SEQ ID NO: 30, a heavy chain CDR3 having an amino acid sequence at least about 70% identical to an amino acid sequence set forth as SEQ ID NO: 31. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 29, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 30, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 31. In some embodiments, antibodies comprise at least one of a heavy chain CDR1 having an amino acid sequence 100% identical to an amino acid sequence set forth as SEQ ID NO: 29, a heavy chain CDR2 having an amino acid sequence 100% identical to an amino acid sequence set forth as SEQ ID NO: 30, a heavy chain CDR3 having an amino acid sequence 100% identical to an amino acid sequence set forth as SEQ ID NO: 31.

In some embodiments, antibodies that selectively bind to a complex comprising an HLA-E and VMAPRTLFL (SEQ ID NO: 38) comprise at least one of a heavy chain CDR1 having an amino acid sequence at least about 70% identical to an amino acid sequence set forth as SEQ ID NO: 33, a heavy chain CDR2 having an amino acid sequence at least about 70% identical to an amino acid sequence set forth as SEQ ID NO: 34, a heavy chain CDR3 having an amino acid sequence at least about 70% identical to an amino acid sequence set forth as SEQ ID NO: 35. In some embodiments, antibodies comprise at least one of a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 33, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 34, a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 35. In some embodiments, antibodies comprise at least one of a heavy chain CDR1 having an amino acid sequence 100% identical to an amino acid sequence set forth as SEQ ID NO: 33, a heavy chain CDR2 having an amino acid sequence 100% identical to an amino acid sequence set forth as SEQ ID NO: 34, a heavy chain CDR3 having an amino acid sequence 100% identical to an amino acid sequence set forth as SEQ ID NO: 35.

In some embodiments, antibodies that selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 1, a light chain CDR2 having an amino acid sequence at least about 70% identical to an amino acid sequence set forth as SEQ ID NO: 2, a light chain CDR3 having an amino acid sequence at least about 70% identical to an amino acid sequence set forth as SEQ ID NO: 3, a heavy chain CDR1 having an amino acid sequence at least about 70% identical to an amino acid sequence set forth as SEQ ID NO: 4, a heavy chain CDR2 having an amino acid sequence at least about 70% identical to an amino acid sequence set forth as SEQ ID NO: 5, and a heavy chain CDR3 having an amino acid sequence at least about 70% identical to an amino acid sequence set forth as SEQ ID NO: 6. In some embodiments, antibodies comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 1, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 2, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 3, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 4, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 5, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 6. In some embodiments, antibodies comprise at least one of a light chain CDR1 having an amino acid sequence 100% identical to an amino acid sequence set forth as SEQ ID NO: 1, a light chain CDR2 having an amino acid sequence 100% identical to an amino acid sequence set forth as SEQ ID NO: 2, a light chain CDR3 having an amino acid sequence 100% identical to an amino acid sequence set forth as SEQ ID NO: 3, a heavy chain CDR1 having an amino acid sequence 100% identical to an amino acid sequence set forth as SEQ ID NO: 4, a heavy chain CDR2 having an amino acid sequence 100% identical to an amino acid sequence set forth as SEQ ID NO: 5, and a heavy chain CDR3 having an amino acid sequence 100% identical to an amino acid sequence set forth as SEQ ID NO: 6.

In some embodiments, antibodies that selectively bind to a complex comprising an HLA-E and VMAPRTLFL (SEQ ID NO: 38) comprise at least one of a light chain CDR1 having an amino acid sequence at least about 70% identical to an amino acid sequence set forth as SEQ ID NO: 9, a light chain CDR2 having an amino acid sequence at least about 70% identical to an amino acid sequence set forth as SEQ ID NO: 10, a light chain CDR3 having an amino acid sequence at least about 70% identical to an amino acid sequence set forth as SEQ ID NO: 11, a heavy chain CDR1 having an amino acid sequence at least about 70% identical to an amino acid sequence set forth as SEQ ID NO: 12, a heavy chain CDR2 having an amino acid sequence at least about 70% identical to an amino acid sequence set forth as SEQ ID NO: 13, and a heavy chain CDR3 having an amino acid sequence at least about 70% identical to an amino acid sequence set forth as SEQ ID NO: 14. In some embodiments, antibodies comprise at least one of a light chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 9, a light chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 10, a light chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 11, a heavy chain CDR1 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 12, a heavy chain CDR2 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 13, and a heavy chain CDR3 having an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 14. In some embodiments, antibodies comprise at least one of a light chain CDR1 having an amino acid sequence 100% identical to an amino acid sequence set forth as SEQ ID NO: 9, a light chain CDR2 having an amino acid sequence 100% identical to an amino acid sequence set forth as SEQ ID NO: 10, a light chain CDR3 having an amino acid sequence 100% identical to an amino acid sequence set forth as SEQ ID NO: 11, a heavy chain CDR1 having an amino acid sequence 100% identical to an amino acid sequence set forth as SEQ ID NO: 12, a heavy chain CDR2 having an amino acid sequence 100% identical to an amino acid sequence set forth as SEQ ID NO: 13, and a heavy chain CDR3 having an amino acid sequence 100% identical to an amino acid sequence set forth as SEQ ID NO: 14.

Disclosed herein are antibodies that selectively bind to a complex comprising an HLA-E and VMAPRTLFL (SEQ ID NO: 38) having a light chain comprising a light chain variable domain (VL). In some embodiments, antibodies comprise a light chain variable domain (VL) having an amino acid sequence at least about 70% identical to an amino acid sequence set forth as SEQ ID NO: 7, or SEQ ID NO: 15. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, or SEQ ID NO: 15. In some embodiments, the VL has an amino acid sequence 100% identical to an amino acid sequence set forth as SEQ ID NO: 7, or SEQ ID NO: 15.

Further disclosed herein are antibodies that selectively bind to a complex comprising an HLA-E and VMAPRTLFL (SEQ ID NO: 38) having a heavy chain comprising a heavy chain variable domain (VH). In some embodiments, antibodies comprise a heavy chain variable domain (VH) having an amino acid sequence at least about 70% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments, the VH has an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

Also disclosed herein are antibodies that selectively bind to a complex comprising an HLA-E and VMAPRTLFL (SEQ ID NO: 38) comprising a light chain variable domain (VL) and a heavy chain variable domain (VH). In some embodiments, antibodies comprise a light chain variable domain (VL) having an amino acid sequence at least about 70% identical to an amino acid sequence set forth as SEQ ID NO: 7, or SEQ ID NO: 15 and a heavy chain variable domain (VH) having an amino acid sequence at least about 70% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7, or SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37. In some embodiments, the VL has an amino acid sequence 100% identical to an amino acid sequence set forth as SEQ ID NO: 7, or SEQ ID NO: 15 and the VH has an amino acid sequence 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

In some embodiments, antibodies that selectively bind to a complex comprising an HLA-E and VMAPRTLFL (SEQ ID NO: 38) comprise a light chain variable domain (VL) having an amino acid sequence at least about 70% identical to an amino acid sequence set forth as SEQ ID NO: 7 and a heavy chain variable domain (VH) having an amino acid sequence at least about 70% identical to an amino acid sequence set forth as SEQ ID NO: 8. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 7 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 8. In some embodiments, the VL has an amino acid sequence 100% identical to an amino acid sequence set forth as SEQ ID NO: 7 and the VH has an amino acid sequence 100% identical to an amino acid sequence set forth as SEQ ID NO: 8.

In some embodiments, antibodies that selectively bind to a complex comprising an HLA-E and VMAPRTLFL (SEQ ID NO: 38) comprise a light chain variable domain (VL) having an amino acid sequence at least about 70% identical to an amino acid sequence set forth as SEQ ID NO: 15 and a heavy chain variable domain (VH) having an amino acid sequence at least about 70% identical to an amino acid sequence set forth as SEQ ID NO: 16. In some embodiments the VL has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 15 and the VH has an amino acid sequence at least about 75%, 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth as SEQ ID NO: 16. In some embodiments, the VL has an amino acid sequence 100% identical to an amino acid sequence set forth as SEQ ID NO: 15 and the VH has an amino acid sequence 100% identical to an amino acid sequence set forth as SEQ ID NO: 16.

In some embodiments, the antibodies selectively bind to a complex comprising a non-classical HLA-I and a neoantigen. In some instances, the antibody does not have a binding affinity to the non-classical HLA-I alone. In some instances, the antibody does not have a binding affinity to the neoantigen alone. In some instances, the antibody does not have a binding affinity to a complex comprising the non-classical HLA-I and a non-relevant neoantigen.

In some instances, the neoantigen is expressed by an antigen processing machinery (APM)-proficient cell. In some instances, the neoantigen is expressed by a TAP1/2-proficient cell. In some instances, the neoantigen is expressed by an antigen processing machinery (APM)-deficient cell. In some instances, the neoantigen comprises, consisting essentially of, or consisting of a sequence according to SEQ ID NO: 38 (VMAPRTLFL).

In some instances, the non-classical HLA-I is HLA-E, HLA-F, HLA-G, or HLA-H. In some instances, the non-classical HLA-I is HLA-E. In some instances, the HLA-E is HLA-E*0101. In some instances, the HLA-E is HLA-E*0103.

In some instances, the antibody selectively binds to the complex comprising the HLA-E and the neoantigen. In some instances, the antibody selectively binds to the complex comprising the HLA-E*0101 and the neoantigen. In some instances, the antibody selectively binds to the complex comprising the HLA-E*0103 and the neoantigen. In some instances, the antibody selectively binds to the complex comprising the HLA-E*0101 and the neoantigen, and to the complex of the HLA-E*0103 and the neoantigen. In some instances, the complex comprises the HLA-E and VMAPRTLFL (SEQ ID NO: 38).

In some instances, the antibody is a murine antibody. In some instances, the antibody is a chimeric antibody. In some instances, the antibody is a camelid antibody. In some instances, the antibody is a humanized antibody. In some instances, the antibody is a human antibody. In some instances, the antibody is a TCR-like antibody. In some instances, the antibody is a single domain antibody. In some instances, the single domain antibody is a camelid single domain antibody. In some instances, the antibody is a multispecific antibody. In some instances, the antibody is a multifunctional antibody.

In some instances, the antibody further comprises a conjugated therapeutic moiety. In some instances, the selective binding of the antibody to the complex comprising the non-classical HLA-I and the neoantigen induces an immune response. In some instances, the immune response comprises activation of T cells. In some instances, the T cell is a CD8+ T cell. In some instances, the immune response comprises activation of cytotoxic T cells (CTLs).

In some instances, the antibody is administered continuously for 1, 2, 3, 4, 5, 6, 7, 8, 9, 10, 14, 15, 28, 30 or more days. In some instances, the antibody is administered at predetermined time intervals for 1, 2, 3, 4, 5, 6, 7, 8, 9, 10, 14, 15, 28, 30 or more days. In some instances, the antibody is administered is administered intermittently for 1, 2, 3, 4, 5, 6, 7, 8, 9, 10, 14, 15, 28, 30 or more days. In some instances, the antibody is administered in 1 dose, 2 doses, 3 doses, 4 doses, 5 doses, 6 doses or more. In some instances, the antibody is administered at a therapeutically effective amount.

In some instances, the cancer is breast cancer. In some instances, the cancer is kidney cancer. In some instances, the cancer is lung cancer. In some instances, the cancer is ovarian cancer. In some instances, the cancer is colorectal cancer. In some instances, the cancer is a B-cell malignancy.

Any suitable route of administration is contemplated for use with the methods disclosed herein. In some embodiments, the antibody is administered by intravenous administration. In some embodiments, the antibody is administered by subcutaneous administration. In some embodiments, the antibody is administered locally. In some embodiments, the antibody is administered systemically (e.g., intravenously, intramuscularly, subcutaneously, intradermally, orally, intranasally, sublingually). In some embodiments, the antibody is formulated as a salve, lotion or emulsion. In some embodiments, the antibody is formulated as a solution. In some embodiments, the antibody is formulated for topical, oral, buccal, or nasal administration.

In some embodiments, the individual is monitored prior to administration of the antibody. Symptoms are identified and their severity is assessed. An antibody as described herein is administered alone or in combination with additional treatments, singly or multiply over time as discussed herein or known to one of skill in the art. In some embodiments, the individual is monitored such that the efficacy of the treatment regimen is determined. In some embodiments, a treatment regimen is modified in response to preliminary treatment outcomes, such that treatment dose or frequency or dose and frequency is altered so as to attain a desired level of subject response in light of symptom alleviation, side effect reduction, or a combination of symptom alleviation and side effect reduction.

Therapeutically effective amounts or dosages are contemplated to include dosages of about 0.01 mg/kg to about 20 mg/kg, about for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6 mg/kg, about 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8 mg/kg, about 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11 mg/kg, about 11.1 mg/kg, about 11.2 mg/kg, about 11.3 mg/kg, about 11.4 mg/kg, about 11.5 mg/kg, about 11.6 mg/kg, about 11.7 mg/kg, about 11.8 mg/kg, about 11.9 mg/kg, about 12 mg/kg, about 12.1 mg/kg, about 12.2 mg/kg, about 12.3 mg/kg, about 12.4 mg/kg, about 12.5 mg/kg, about 12.6 mg/kg, about 12.7 mg/kg, about 12.8 mg/kg, about 12.9 mg/kg, about 13 mg/kg, about 13.1 mg/kg, about 13.2 mg/kg, about 13.3 mg/kg, about 13.4 mg/kg, about 13.5 mg/kg, about 13.6 mg/kg, about 13.7 mg/kg, about 13.8 mg/kg, about 13.9 mg/kg, about 14 mg/kg, about 14.1 mg/kg, about 14.2 mg/kg, about 14.3 mg/kg, about 14.4 mg/kg, about 14.5 mg/kg, about 14.6 mg/kg, about 14.7 mg/kg, about 14.8 mg/kg, about 14.9 mg/kg, about 15 mg/kg, about 15.1 mg/kg, about 15.2 mg/kg, about 15.3 mg/kg, about 15.4 mg/kg, about 15.5 mg/kg, about 15.6 mg/kg, about 15.7 mg/kg, about 15.8 mg/kg, about 15.9 mg/kg, about 16 mg/kg, about 16.1 mg/kg, about 16.2 mg/kg, about 16.3 mg/kg, about 16.4 mg/kg, about 16.5 mg/kg, about 16.6 mg/kg, about 16.7 mg/kg, about 16.8 mg/kg, about 16.9 mg/kg, about 17 mg/kg, about 17.1 mg/kg, about 17.2 mg/kg, about 17.3 mg/kg, about 17.4 mg/kg, about 17.5 mg/kg, about 17.6 mg/kg, about 17.7 mg/kg, about 17.8 mg/kg, about 17.9 mg/kg, about 18 mg/kg, about 18.1 mg/kg, about 18.2 mg/kg, about 18.3 mg/kg, about 18.4 mg/kg, about 18.5 mg/kg, about 18.6 mg/kg, about 18.7 mg/kg, about 18.8 mg/kg, about 18.9 mg/kg, about 19 mg/kg, about 19.1 mg/kg, about 19.2 mg/kg, about 19.3 mg/kg, about 19.4 mg/kg, about 19.5 mg/kg, about 19.6 mg/kg, about 19.7 mg/kg, about 19.8 mg/kg, about 19.9 mg/kg, about or 20 mg/kg. Therapeutically effective amounts or dosages, in some cases, are contemplated to include dosages of about 0.1 mg/kg to about 2.0 mg/kg.

Methods of treatment herein comprise one or more administrations of antibodies in doses disclosed herein. In some embodiments, methods comprise one administration of antibodies. In some embodiments, methods comprise two administrations of antibodies. In some embodiments, methods comprise three administrations of antibodies. In some embodiments, methods comprise four administrations of antibodies. In some embodiments, methods comprise five administrations of antibodies. In some embodiments, methods comprise six administrations of antibodies. In some embodiments, one or more administrations of antibodies are administered daily. In some embodiments, one or more administrations of antibodies are administered weekly. In some embodiments, one or more administrations of antibodies are administered biweekly. In some embodiments, one or more administrations of antibodies are administered monthly. In some embodiments, one or more administrations of antibodies are administered every three months. In some embodiments, one or more administrations of antibodies are administered every six months. In some embodiments, one or more administrations of antibodies are administered yearly.

### Pharmaceutical Compositions

Also disclosed herein are pharmaceutical compositions comprising antibodies that selectively bind to a complex comprising a non-classical HLA-I (e.g. HLA-E) and a neoantigen (VMAPRTLFL (SEQ ID NO: 38)) disclosed herein and a pharmaceutically acceptable carrier or excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments, the compositions further comprise an additional therapeutic agent. In some embodiments, the therapeutic agent is a chemotherapeutic agent. The chemotherapeutic agents can include, among others, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In some embodiments, the antibody and the therapeutic agent are in the same formulation. In some embodiments, the antibody and the therapeutic agent are in different formulation. In some embodiments, antibody described herein is used prior to the administration of the other therapeutic agent. In some embodiments, antibody described herein is used concurrently with the administration of the other therapeutic agent. In some embodiments, antibody described herein is used subsequent to the administration of the other therapeutic agent.

Pharmaceutical formulations, in some embodiments, are made to be compatible with a particular local, regional or systemic administration or delivery route. Thus, pharmaceutical formulations include carriers, diluents, or excipients suitable for administration by particular routes. Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

### Therapeutic Regimens for a Pharmaceutical Composition

In some embodiments, pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, in some instances, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular composition, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the composition used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compositions exhibiting high therapeutic indices are preferred. The data obtained from cell culture assays and animal studies are used in formulating a range of dosage for use in human. The dosage of such composition lies preferably within a range of circulating concentrations that include the ED50 with minimal toxicity. The dosage varies within this range depending upon the dosage form employed and the route of administration utilized.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Discovery of Antibodies, MAB-031 and MAB-036 that Specifically Target the HLA-E/VMAPRTLFL Peptide Complex

Phage display and yeast display technology was used to identify scFv binders. Briefly, to generate the antibody, MAB-031, Balb/c mice were immunized 3× with 50 ug/injection of antigen, HLA-E/VMAPRTLFL peptide complex. One week after final injection. Sera from immunized mice was collected and tested for antibody response by ELISA. The titer reached in several of the immunized mice was greater than 1:100,000 and the spleen from the best responsive mouse was removed and used to construct the scFv antibody library in phage. Total RNA was isolated using the TriZol methods and RNA was then assessed by gel electrophoresis.

PCR amplification was performed next. In brief, VH and VL genes were amplified from cDNA template using murine specific primers. The scFv cassettes were assembled by over-lapping PCR. scFv genes and phagemid (pHENI) were digested using restriction enzymes and ligated together with T4 DNA ligase. The ligation mix was desalted and re-suspended in distilled water before being used to electro-transform TG1 E. coli competent cells to construct final library. Finally, phage displaying scFv proteins were packaged with the aid of helper phage M13Ko7 following standard methods.

In the first two rounds of biopanning, depletion was carried out using a biotin-labeled HLA-A2-peptide mix to remove and prevent binding of non-specific scFv expressing phage in the library. Following this step, positive panning for the target using biotin-labeled HLA-E-VMAPRTLFL was performed. In parallel, panning of same immune scFv phage library against groups with no coating (no antigen) and coating with biotin-labeled HLA-A2-peptide was performed. The third round of panning was performed next using biotin-labeled HLA-E-YLLPAIVHI for depletion and pre-blocking. After that, positive panning for biotin-labeled HLA-E-VMAPRTLFL was performed. In parallel the library was again panned against two control groups: no coating and coating with biotin-labeled HLA-E-YLLPAIVHI. Enrichment was observed between the target group and control screening groups. 40 clones were selected from the third round of elution output to validate the specificity of enrichment, 13 clones bound to positive target HLA-E-VMAPRTLFL and 6 clones from that group had unique sequence. The scFv clone used to produce MAB-031 was identified from the group of 6 clones as having good specificity to the HLA-E-VMAPRTLFL target.

The scFv binder that led to the generation of MAB-036 was originally derived from a pre-made human antibody library. A phage library using the monodisplay by pIX fusion was constructed in the scFv format using semi-synthesized VH and VL genes to create a total diversity of 1.42×10⁹. The library was propagated using E. coli TG1 host strain along with M13K07 helper phage. The enriched scFv clones from the first round of phage panning were amplified and cloned into the yeast plasmid containing a c-terminus FLAG tag. Four rounds of selection followed and the scFv clone R4, specific for HLA-E-VMAPRTLFL was identified. The scFv R4 antibody was then cloned into yeast and the affinity of the antibody was further optimized by introducing random mutations into the CDR3H region resulting in the identification of clone #1. Next, the CDR2L of R4 clone #1 was mutated to remove a potential glycosylation site. This clone was named R4Clone #1 mutated light chain and was further optimized by grafting the CDR from both H and L chains onto the VH and VL framework regions of trastuzumab. This modification led to a significant improvement in antibody stability and the VH and VL domains were cloned into the pFUSE Vector system to produce full-length human IgG1 antibodies. The full-length antibody clone was named MAB-036.

### Example 2. Assessing the Binding Specificity and Sensitivity of MAB-031 and MAB-036 to Target HLA-E/VMAPRTLFL by ELISA

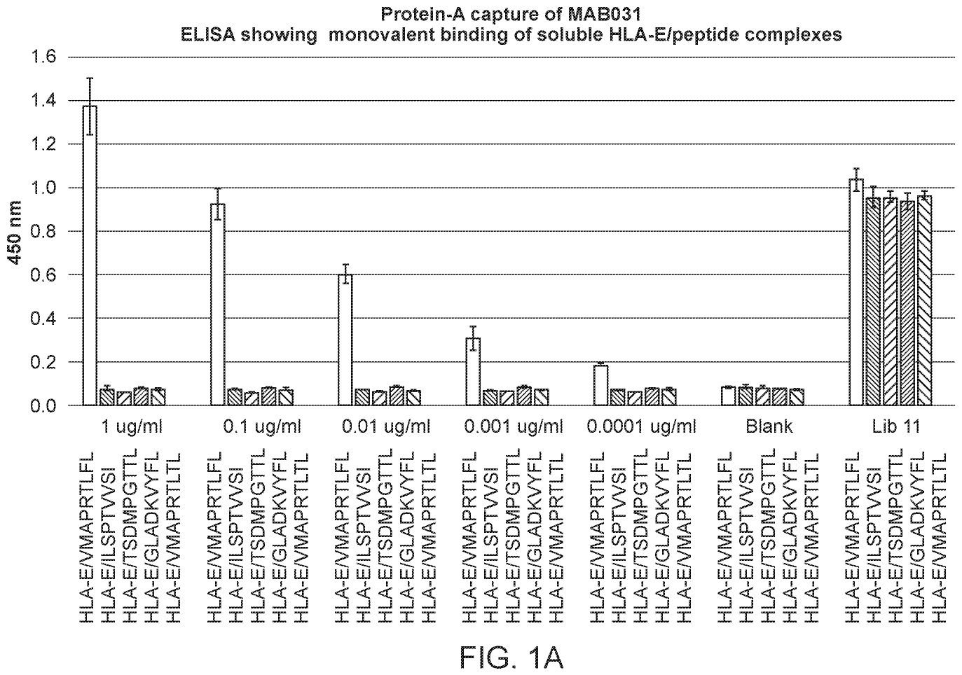

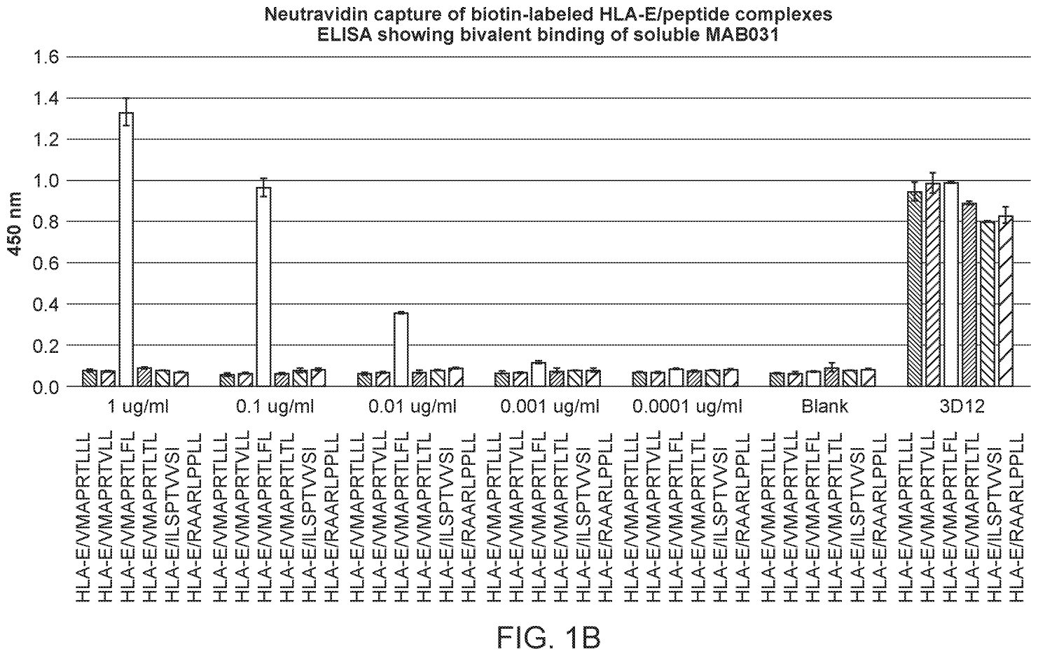

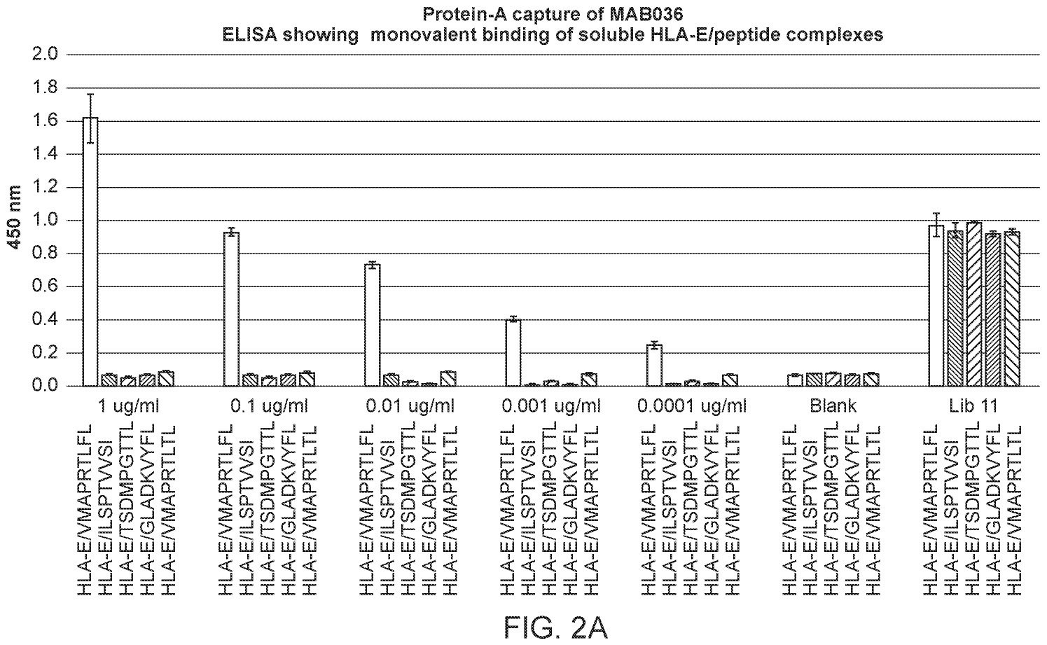

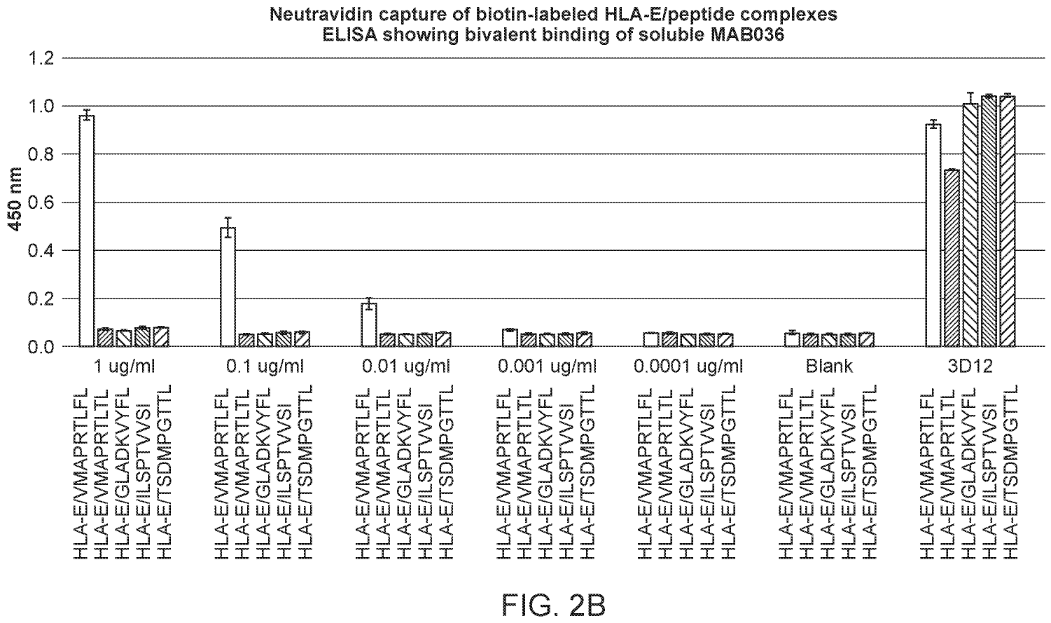

The isolated scFv VL and VH domains were cloned into pFUSE-hIgG1-Fc and pFUSE2-CLIg-hk plasmids, respectively and plasmid DNA was prepared using standard protocol and used to co-transfect a 200 ml culture of Expi-293 cells. Following the manufacturer's instructions, supernatants were harvested at day 5 post-transfection for antibody purification using Protein-A coated beads. Next, the purified mouse-human chimeric IgG1 antibody, MAB-031 and the fully human antibody, MAB-036 were characterized for binding specificity and sensitivity by ELISA. In brief, Protein-A coated 96-wells were used to capture either MAB-031 or MAB-036 added to wells at a concentration of 10 ug/ml. After incubation for 60 min, wells were washed using PBS (pH 7.4)+0.1% Tween-20 and soluble biotin-labeled HLA-E/peptide complexes were added to individual wells at concentrations ranging from 0.25 to 0.0625 ug/ml. Wells were washed 3× using a solution of PBS+Tween-20 and then streptavidin-Horseradish peroxidase (SA-HRP) conjugate was added to wells and incubated for 30 min. One final wash was done and the substrate solution containing 3,3′,5,5′-tetramethylbenzidine (TMB) was added to wells and developed for 10 min before adding stop solution (0.16M sulfuric acid) to terminate the reaction. ELISA results using antibodies, MAB-031 and MAB-036 are shown in FIG. 1A and FIG. 2A. Both MAB-031 and MAB-036 displayed high selectivity for the HLA-E/VAMPRTLFL target and exhibited no cross-reactivity for the control HLA-E/peptide complexes, HLA-E/VMAPRTVTL, HLA-E/ILSPTVVSI, HLA-E/TSDMPGTTL, and HLA-E/GLADKVYF. In addition, the both MABs displayed strong binding to soluble biotinylated HLA-E/VMAPRTLFL complexes even at 0.0001 ug/ml, the lowest concentration tested.

To further evaluate the binding specificity and detection sensitivity of MAB-031 and MAB-036, a second ELISA was performed. For this assay, neutravidin coated plates were used to capture biotin-labeled HLA-E/peptide complexes. Because the HLA-E/peptide complexes have a single biotin molecule conjugated to the c-terminal end BirA tag, the immobilization of these complexes results in having the molecules lined up in a homogenous directional orientation. After incubation, unbound biotin-labeled HLA-E/peptide complexes are removed by rinsing the plate in a PBS+tween-20 buffer solution. MAB31 and MAB36 were then added to wells at concentrations that ranged from 1 ug/ml to 0.0001 ug/ml. Incubation was carried out for 1 hr, wells were rinsed and bound MAB was detected using a 1:2500 dilution of a goat anti-human-HRP conjugate. The assay was developed by adding the TMB substrate solution was added to wells and developed for 10 min before adding stop solution to terminate the reaction. ELISA results using antibodies, MAB-031 and MAB-036 are shown in FIG. 1B and FIG. 2B. Both MAB-031 and MAB-036 used a bivalent antibody displayed high selectivity for the HLA-E/VAMPRTLFL target and exhibited no cross-reactivity for the control HLA-E/peptide complexes, HLA-E/VMAPRTLLL, HLA-E/VMAPRTVLL, HLA-E/ILSPTVVSI, HLA-E/RAARLPPLL, and HLA-E/VMAPRTLTL. In addition, both MABs displayed strong binding to immobilized HLA-E/VMAPRTLFL complexes.

### Example 3. Detection of HLA-E/VMAPRTLFL Complexes on Tumor Cells Using MAB-031 and MAB-036

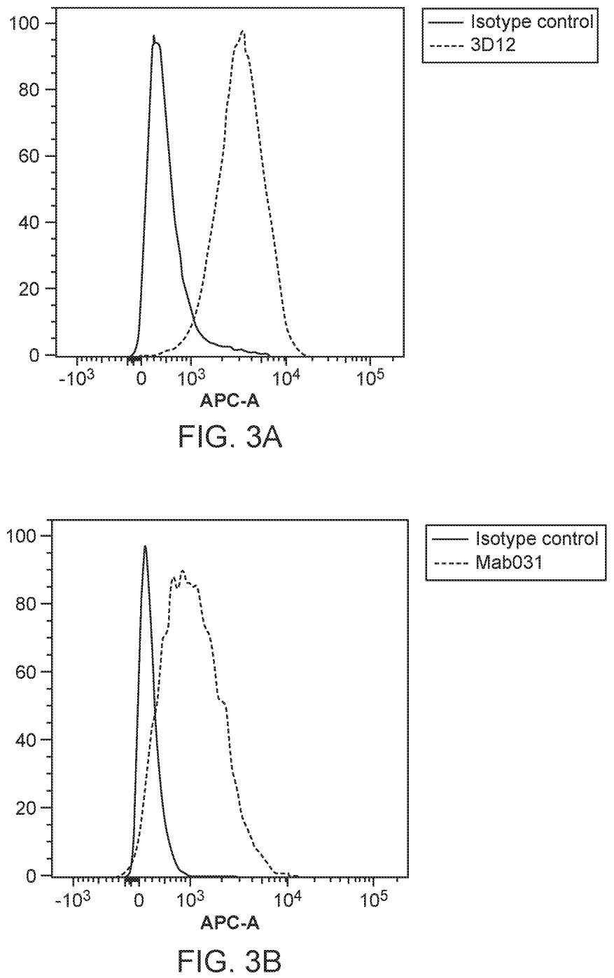

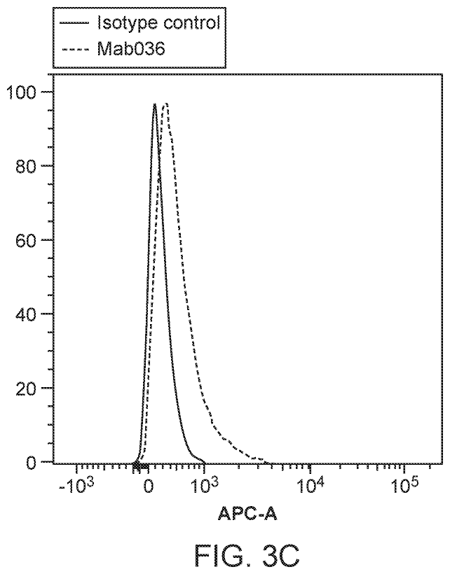

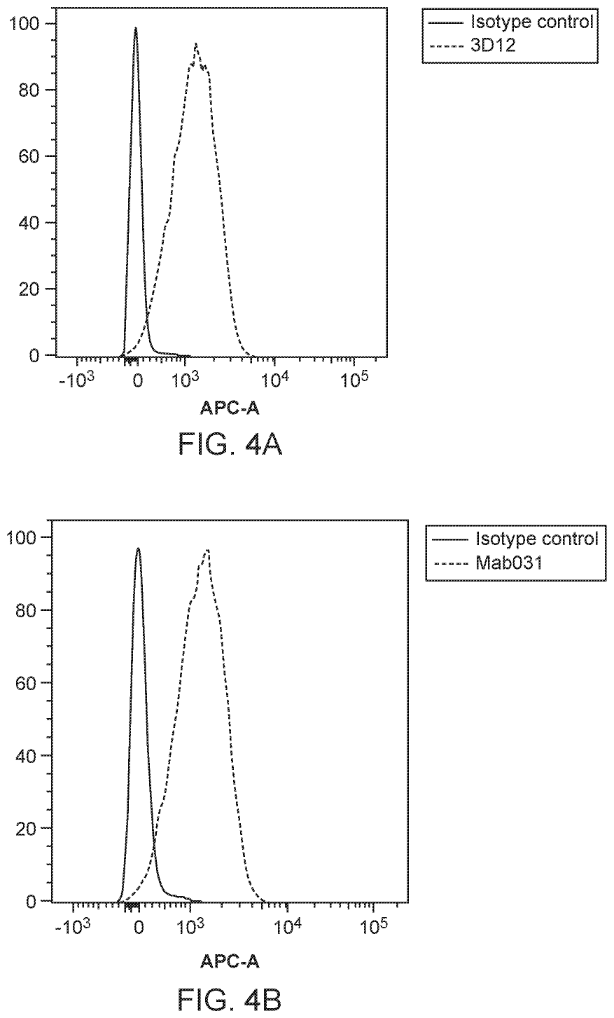

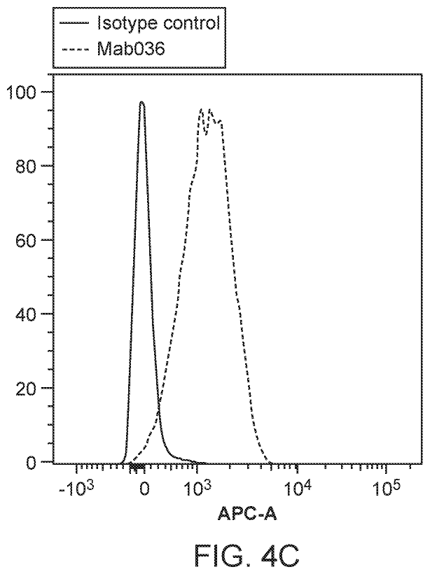

The next study was to test the ability of MAB-31 and MAB-036 to detect the HLA-E/VMAPRTLFL target on tumor cells. For these studies two cell lines were selected: JEG-3, a human placenta choriocarcinoma cell line and JVM-2, a human Mantle lymphoma. Both cells express high levels of HLA-E and HLA-G. Detection of HLA-E surface expression was demonstrated using the murine antibody, 3D12-APC conjugate and flow cytometry (FIG. 3A and FIG. 4A). HLA-G expression for each cell line was shown either using the antibody MEM-G/9 and flow cytometry or performing western blot analysis on cell lysis using the anti-HLA-G antibody, 87G. MAB=−31 and MAB-036 were used at 1 ug/ml concentration to stain JEG-3 and JVM-2 cells. Following incubation of cells with each MAB, cells were washed in PBS containing 0.5% BSA and 2 mM EDTA (wash buffer). Detection of bound antibody was carried out using a goat anti-human IgG1-APC labeled conjugate. Cells were rinsed again in wash buffer and then run on a BD LSR II flow cytometer. Data analysis was performed using Flowjo V10. Strong staining of MAB-031 was observed for both cell lines though MAB-036 revealed slightly weaker staining for JEG-3 cells. (FIG. 3B-FIG. 3C, and FIG. 4B-FIG. 4C).

### Example 4. Absence of Antibody Binding to Control Cells

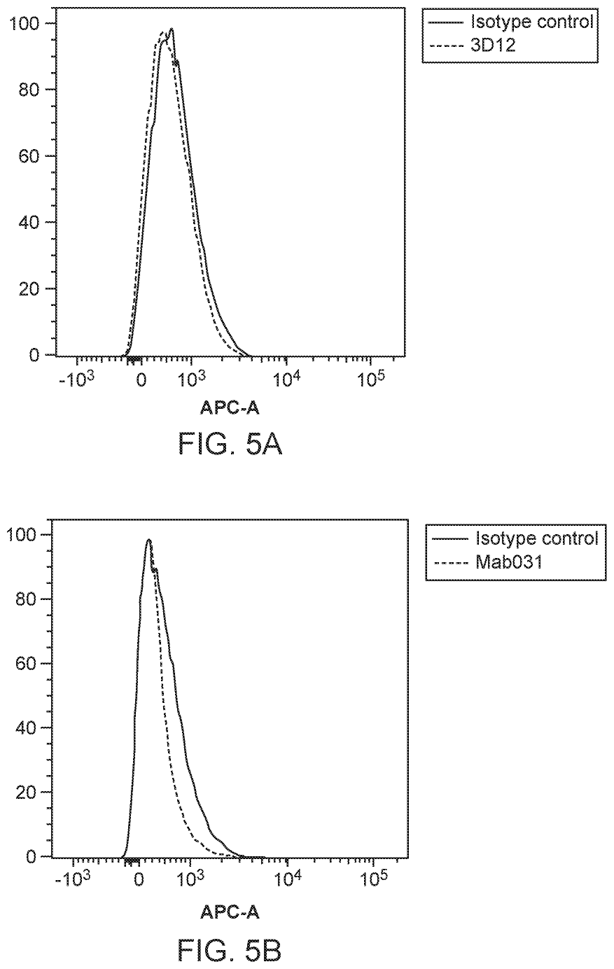

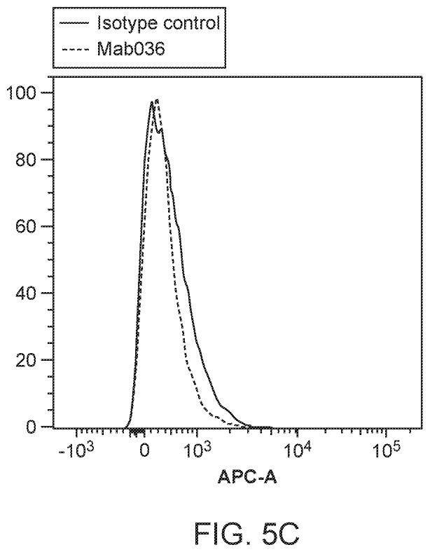

To assess the binding specificity and potential cross-reactivity of antibodies generated to the HLA-E/VMAPRTLFL complex, MAB-031 and MAB-036 were selected for evaluation using the antibodies at 1 ug/ml concentration to stain A549 human lung cancer cells. A549 cancer cells do not express HLA-E as determined by a lack of 3D12-APC (0.5 ug/ml) antibody conjugate staining (FIG. 5A). The A549 cells were stained with 1.0 ug/ml of MAB-031 and MAB-036 for 1 hr followed by cells being washed with PBS containing 0.5% bovine serum albumin (BSA) and 2 m MEDTA. Detection of bound antibody was carried out using goat anti-human IgG1-APC labeled conjugate. Cells were washed again in same buffer and then run on a BD LSR II flow cytometer. Data analysis was performed using Flowjo V10. Software. As anticipated, MAB-031 and MAB-036 did not stain A549 cells (FIG. 5B-FIG. 5C).

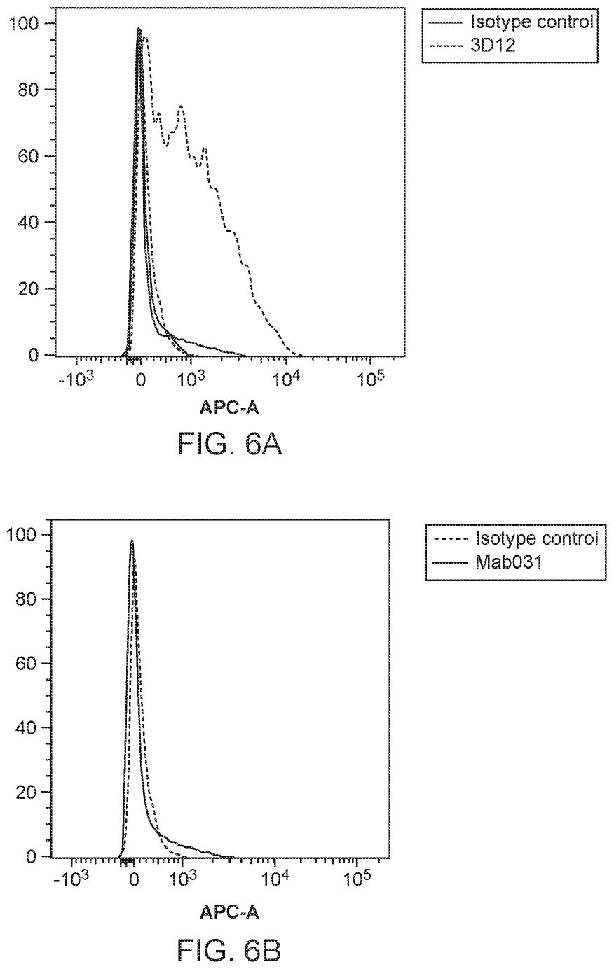

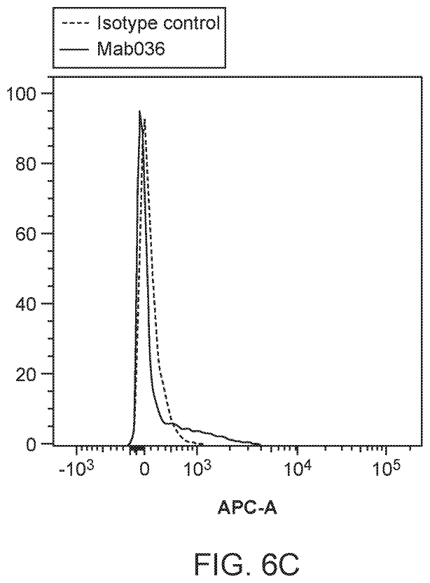

Next, MAB-031 and MAB-036 were used to stain the tumor cell line EB-1 (Burkitt's lymphoma). This tumor cell line expresses high levels of HLA-E detected using 3D12-APC antibody conjugate (FIG. 6A). MAB-031 and MAB-036 were used at 1 ug/ml concentration to stain EB-1 cells. Following incubation of cells with the four MABs, cells were washed in PBS containing 0.5% BSA and 2mMEDTA (wash buffer). Detection of bound antibody was carried out using a goat anti-human IgG1-APC labeled conjugate. Cells were rinsed again in wash buffer and then run on a BD LSR II flow cytometer. Data analysis was performed using Flowjo V10. No detectable binding to EB-1 cells was observed with either antibody (FIG. 6B-FIG. 6C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A monoclonal antibody or an antigen-binding fragment thereof, comprising a light chain variable domain (VL) comprising an amino acid sequence at least 80% identical to an amino acid sequence set forth as SEQ ID NO: 7, or SEQ ID NO: 15.

2. The monoclonal antibody of claim 1, wherein the light chain variable domain (VL) comprises an amino acid sequence at least 90%, at least 95%, at least 99% or 100% identical to an amino acid sequence set forth as SEQ ID NO: 7, or SEQ ID NO: 15.

3. The monoclonal antibody of claim 1, wherein the monoclonal antibody or antigen-binding fragment thereof comprises a heavy chain variable domain (VH) comprising an amino acid sequence at least 80%, at least 90%, at least 95%, at least 99%, or 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

4. The monoclonal antibody of claim 1, wherein the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8.

5. The monoclonal antibody of claim 1, wherein the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16.

6. The monoclonal antibody of claim 1, wherein the monoclonal antibody or antigen-binding fragment thereof selectively binds to a complex comprising an HLA-E and a neoantigen.

7. The monoclonal antibody of claim 1, wherein the monoclonal antibody or antigen-binding fragment thereof does not have a binding affinity to (i) the HLA-E alone; or (ii) the neoantigen alone.

8. The monoclonal antibody of claim 1, wherein the neoantigen comprises, consisting essentially of, or consisting of a sequence according to SEQ ID NO: 38 (VMAPRTLFL).

9. The monoclonal antibody of claim 1, wherein the complex comprises the HLA-E and VMAPRTLFL (SEQ ID NO: 38).

10. The monoclonal antibody of claim 1, wherein the monoclonal antibody or antigen-binding fragment thereof is a murine antibody, a chimeric antibody, a camelid antibody, a humanized antibody, or a human antibody.

11. The monoclonal antibody of claim 1, wherein the monoclonal antibody or antigen-binding fragment thereof is a TCR-like antibody.

12. The monoclonal antibody of claim 1, wherein the monoclonal antibody or antigen-binding fragment thereof antibody further comprises a conjugated therapeutic moiety.

13. The monoclonal antibody of claim 1, wherein the selective binding of the antibody to the complex comprising the HLA-E and the neoantigen induces an immune response in a cell.

14. The monoclonal antibody of claim 13, wherein the cell is a cancer cell.

15. A monoclonal antibody or an antigen-binding fragment thereof, comprising a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

16. The monoclonal antibody of claim 15, wherein the heavy chain variable domain (VH) comprises an amino acid sequence at least 90%, at least 95%, at least 99%, or 100% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

17. The monoclonal antibody of claim 15, wherein the monoclonal antibody or antigen-binding fragment thereof comprises a light chain variable domain (VL) comprising an amino acid sequence at least 80%, at least 90%, at least 95%, at least 99%, or 100% identical to an amino acid sequence set forth as SEQ ID NO: 7, or SEQ ID NO: 15.

18. The monoclonal antibody of claim 15, wherein the monoclonal antibody or antigen-binding fragment thereof comprises a heavy chain variable domain (VH) comprising an amino acid sequence at least 80% identical to SEQ ID NO: 8 and a light chain variable domain (VL) comprising an amino acid sequence at least 80% identical to SEQ ID NO: 7.

19. The monoclonal antibody of claim 15, wherein the monoclonal antibody or antigen-binding fragment thereof comprises a heavy chain variable domain (VH) comprising an amino acid sequence at least 80% identical to SEQ ID NO: 16 and a light chain variable domain (VL) comprising an amino acid sequence at least 80% identical to SEQ ID NO: 15.

20. The monoclonal antibody of claim 15, wherein the monoclonal antibody or antigen-binding fragment thereof selectively binds to a complex comprising an HLA-E and a neoantigen.

21. The monoclonal antibody of claim 15, wherein the monoclonal antibody or antigen-binding fragment thereof does not have a binding affinity to (i) the HLA-E alone; or (ii) the neoantigen alone.

22. The monoclonal antibody of claim 15, wherein the neoantigen comprises, consisting essentially of, or consisting of a sequence according to SEQ ID NO: 38 (VMAPRTLFL).

23. The monoclonal antibody of claim 15, wherein the complex comprises the HLA-E and VMAPRTLFL (SEQ ID NO: 38).

24. The monoclonal antibody of claim 15, wherein the monoclonal antibody or antigen-binding fragment thereof is a murine antibody, a chimeric antibody, a camelid antibody, a humanized antibody, or a human antibody.

25. The monoclonal antibody of claim 15, wherein the monoclonal antibody or antigen-binding fragment thereof is a TCR-like antibody.

26. The monoclonal antibody of claim 15, wherein the monoclonal antibody or antigen-binding fragment thereof antibody further comprises a conjugated therapeutic moiety.

27. The monoclonal antibody of claim 15, wherein the selective binding of the antibody to the complex comprising the HLA-E and the neoantigen induces an immune response in a cell.

28. The monoclonal antibody of claim 27, wherein the cell is a cancer cell.

29. A monoclonal antibody or an antigen-binding fragment thereof, comprising a light chain complementarity determining region (CDR) having an amino acid sequence at least 80% identical to at least one of the amino acid sequences set forth as SEQ ID NOS: 1-3, or 9-11.

30. The monoclonal antibody of claim 29, wherein the light chain complementarity determining region (CDR) has an amino acid sequence at least 90%, at least 95%, at least 99%, or 100% identical to at least one of the amino acid sequences set forth as SEQ ID NOS: 1-3, or 9-11.

31. The monoclonal antibody of claim 29, wherein the monoclonal antibody or antigen-binding fragment thereof comprises a heavy chain complementarity determining region (CDR) having an amino acid sequence at least 80%, at least 90%, at least 95%, at least 99%, or 100% identical to at least one of the amino acid sequences set forth as SEQ ID NOS: 4-6, 12-14, 17-19, 21-23, 25-27, 29-31, and 33-35.

32. The monoclonal antibody claim 29, wherein the monoclonal antibody or antigen-binding fragment thereof comprises a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35.

33. The monoclonal antibody of claim 29, wherein the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3.

34. The monoclonal antibody of claim 29, wherein the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11.

35. The monoclonal antibody of claim 29, wherein the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6.

36. The monoclonal antibody of claim 29, wherein the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14.

37. The monoclonal antibody of claim 29, wherein the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15.

38. The monoclonal antibody of claim 29, wherein the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

39. The monoclonal antibody of claim 29, wherein the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8.

40. The monoclonal antibody of claim 29, wherein the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16.

41. The monoclonal antibody of claim 29, wherein the monoclonal antibody or antigen-binding fragment thereof selectively binds to a complex comprising an HLA-E and a neoantigen.

42. The monoclonal antibody of claim 29, wherein the monoclonal antibody or antigen-binding fragment thereof does not have a binding affinity to (i) the HLA-E alone; or (ii) the neoantigen alone.

43. The monoclonal antibody of claim 29, wherein the neoantigen comprises, consisting essentially of, or consisting of a sequence according to SEQ ID NO: 38 (VMAPRTLFL).

44. The monoclonal antibody of claim 29, wherein the complex comprises the HLA-E and VMAPRTLFL (SEQ ID NO: 38).

45. The monoclonal antibody of claim 29, wherein the monoclonal antibody or antigen-binding fragment thereof is a murine antibody, a chimeric antibody, a camelid antibody, a humanized antibody, or a human antibody.

46. The monoclonal antibody of claim 29, wherein the monoclonal antibody or antigen-binding fragment thereof is a TCR-like antibody.

47. The monoclonal antibody of claim 29, wherein the monoclonal antibody or antigen-binding fragment thereof antibody further comprises a conjugated therapeutic moiety.

48. The monoclonal antibody of claim 29, wherein the selective binding of the antibody to the complex comprising the HLA-E and the neoantigen induces an immune response in a cell.

49. The monoclonal antibody of claim 48, wherein the cell is a cancer cell.

50. A monoclonal antibody or an antigen-binding fragment thereof, comprising a heavy chain complementarity determining region (CDR) having an amino acid sequence at least 80% identical to at least one of the amino acid sequences set forth as SEQ ID NOS: 4-6, 12-14, 17-19, 21-23, 25-27, 29-31, and 33-35.

51. The monoclonal antibody of claim 50, wherein the heavy chain complementarity determining region (CDR) has an amino acid sequence at least 90%, at least 95%, at least 99%, or 100% identical to at least one of the amino acid sequences set forth as SEQ ID NOS: 4-6, 12-14, 17-19, 21-23, 25-27, 29-31, and 33-35.

52. The monoclonal antibody of claim 50, wherein the monoclonal antibody or antigen-binding fragment comprises a light chain complementarity determining region (CDR) having an amino acid sequence at least 80%, at least 90%, at least 95%, at least 99%, or 100% identical to at least one of the amino acid sequences set forth as SEQ ID NOS: 1-3, and 9-11.

53. The monoclonal antibody of claim 50, wherein the monoclonal antibody or antigen-binding fragment thereof comprises a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35, a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, and a light chain complementarity determining region 3 (CDR3) having an amino acid sequence at least 80% identical to one of SEQ ID NO: 3, or SEQ ID NO: 11.

54. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6.

55. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14.

56. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 17, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 19.

57. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 21, a heavy chain complementarity determining region 2 (CDR2) having an amino acid sequence at least 80% identical to SEQ ID NO: 22, and a heavy chain complementarity determining region 3 (CDR3) having an amino acid sequence at least 80% identical to SEQ ID NO: 23.

58. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 25, a heavy chain complementarity determining region 2 (CDR2) having an amino acid sequence at least 80% identical to SEQ ID NO: 26, and a heavy chain complementarity determining region 3 (CDR3) having an amino acid sequence at least 80% identical to SEQ ID NO: 27.

59. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 29, a heavy chain complementarity determining region 2 (CDR2) having an amino acid sequence at least 80% identical to SEQ ID NO: 30, and a heavy chain complementarity determining region 3 (CDR3) having an amino acid sequence at least 80% identical to SEQ ID NO: 31.

60. The monoclonal antibody of claim 50, wherein the monoclonal antibody or antigen-binding fragment thereof comprises at least one of a heavy chain complementarity determining region 1 (CDR1) having an amino acid sequence at least 80% identical to SEQ ID NO: 33, a heavy chain complementarity determining region 2 (CDR2) having an amino acid sequence at least 80% identical to SEQ ID NO: 34, and a heavy chain complementarity determining region 3 (CDR3) having an amino acid sequence at least 80% identical to SEQ ID NO: 35.

61. The monoclonal antibody of claim 50, wherein the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3.

62. The monoclonal antibody of claim 50, wherein the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11.

63. The monoclonal antibody of claim 50, wherein the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

64. The monoclonal antibody of claim 50, wherein the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15.

65. The monoclonal antibody of claim 50, wherein the monoclonal antibody or antigen-binding fragment thereof comprises a heavy chain variable domain (VH) comprising an amino acid sequence at least 80% identical to SEQ ID NO: 8 and a light chain variable domain (VL) comprising an amino acid sequence at least 80% identical to SEQ ID NO: 7.

66. The monoclonal antibody of claim 50, wherein the monoclonal antibody or antigen-binding fragment thereof comprises a heavy chain variable domain (VH) comprising an amino acid sequence at least 80% identical to SEQ ID NO: 16 and a light chain variable domain (VL) comprising an amino acid sequence at least 80% identical to SEQ ID NO: 15.

67. The monoclonal antibody of claim 50, wherein the monoclonal antibody or antigen-binding fragment thereof selectively binds to a complex comprising an HLA-E and a neoantigen.

68. The monoclonal antibody of claim 50, wherein the monoclonal antibody or antigen-binding fragment thereof does not have a binding affinity to (i) the HLA-E alone; or (ii) the neoantigen alone.

69. The monoclonal antibody of claim 50, wherein the neoantigen comprises, consisting essentially of, or consisting of a sequence according to SEQ ID NO: 38 (VMAPRTLFL).

70. The monoclonal antibody of claim 50, wherein the complex comprises the HLA-E and VMAPRTLFL (SEQ ID NO: 38).

71. The monoclonal antibody of claim 50, wherein the monoclonal antibody or antigen-binding fragment thereof is a murine antibody, a chimeric antibody, a camelid antibody, a humanized antibody, or a human antibody.

72. The monoclonal antibody of claim 50, wherein the monoclonal antibody or antigen-binding fragment thereof is a TCR-like antibody.

73. The monoclonal antibody of claim 50, wherein the monoclonal antibody or antigen-binding fragment thereof antibody further comprises a conjugated therapeutic moiety.

74. The monoclonal antibody of claim 50, wherein the selective binding of the antibody to the complex comprising the HLA-E and the neoantigen induces an immune response in a cell.

75. The monoclonal antibody of claim 74, wherein the cell is a cancer cell.

76. A pharmaceutical composition comprising: a monoclonal antibody or an antigen-binding fragment thereof according to any one of claims 1 to 75; and a pharmaceutically acceptable carrier or excipient.

77. A method of treating cancer in an individual in need thereof, comprising administering to the individual an effective amount of a monoclonal antibody or an antigen-binding fragment thereof comprising a light chain complementarity determining region (CDR) having an amino acid sequence at least 80%, at least 90%, at least 95%, at least 99%, or 100% identical to at least one of the amino acid sequences set forth as SEQ ID NOS: 1-3, or 9-11.

78. The method of claim 77, wherein the monoclonal antibody or an antigen-binding fragment thereof comprises a heavy chain complementarity determining region (CDR) having an amino acid sequence at least 80%, at least 90%, at least 95%, at least 99%, or 100% identical to at least one of the amino acid sequences set forth as SEQ ID NOS: 4-6, 12-14, 17-19, 21-23, 25-27, 29-31, and 33-35.

79. The method of claim 77, wherein the monoclonal antibody or antigen-binding fragment thereof comprises a light chain complementarity determining region 1 (CDR1) having an amino acid sequence at least 80% identical to one of SEQ ID NO: 1, or SEQ ID NO: 9, a light chain complementarity determining region 2 (CDR2) having an amino acid sequence at least 80% identical to one of SEQ ID NO: 2, or SEQ ID NO: 10, a light chain complementarity determining region 3 (CDR3) having an amino acid sequence at least 80% identical to one of SEQ ID NO: 3, or SEQ ID NO: 11, a heavy chain complementarity determining region 1 (CDR1) having an amino acid sequence at least 80% identical to one of SEQ ID NO: 4, SEQ ID NO: 12, SEQ ID NO: 17, SEQ ID NO: 21, SEQ ID NO: 25, SEQ ID NO: 29, or SEQ ID NO: 33, a heavy chain complementarity determining region 2 (CDR2) having an amino acid sequence at least 80% identical to one of SEQ ID NO: 5, SEQ ID NO: 13, SEQ ID NO: 18, SEQ ID NO: 22, SEQ ID NO: 26, SEQ ID NO: 30, or SEQ ID NO: 34, and a heavy chain complementarity determining region 3 (CDR3) having an amino acid sequence at least 80% identical to one of SEQ ID NO: 6, SEQ ID NO: 14, SEQ ID NO: 19, SEQ ID NO: 23, SEQ ID NO: 27, SEQ ID NO: 31, or SEQ ID NO: 35.

80. The method of claim 77, wherein the monoclonal antibody or antigen-binding fragment thereof comprises at least one of a light chain complementarity determining region 1 (CDR1) having an amino acid sequence at least 80% identical to SEQ ID NO: 1, a light chain complementarity determining region 2 (CDR2) having an amino acid sequence at least 80% identical to SEQ ID NO: 2, and a light chain complementarity determining region 3 (CDR3) having an amino acid sequence at least 80% identical to SEQ ID NO: 3.

81. The method of claim 77, wherein the monoclonal antibody or antigen-binding fragment thereof comprises at least one of a light chain complementarity determining region 1 (CDR1) having an amino acid sequence at least 80% identical to SEQ ID NO: 9, a light chain complementarity determining region 2 (CDR2) having an amino acid sequence at least 80% identical to SEQ ID NO: 10, and a light chain complementarity determining region 3 (CDR3) having an amino acid sequence at least 80% identical to SEQ ID NO: 11.

82. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 4, a heavy chain complementarity determining region 2 (CDR2) having an amino acid sequence at least 80% identical to SEQ ID NO: 5, and a heavy chain complementarity determining region 3 (CDR3) having an amino acid sequence at least 80% identical to SEQ ID NO: 6.

83. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 12, a heavy chain complementarity determining region 2 (CDR2) having an amino acid sequence at least 80% identical to SEQ ID NO: 13, and a heavy chain complementarity determining region 3 (CDR3) having an amino acid sequence at least 80% identical to SEQ ID NO: 14.

84. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 17, a heavy chain complementarity determining region 2 (CDR2) having an amino acid sequence at least 80% identical to SEQ ID NO: 18, and a heavy chain complementarity determining region 3 (CDR3) having an amino acid sequence at least 80% identical to SEQ ID NO: 19.

85. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 21, a heavy chain complementarity determining region 2 (CDR2) having an amino acid sequence at least 80% identical to SEQ ID NO: 22, and a heavy chain complementarity determining region 3 (CDR3) having an amino acid sequence at least 80% identical to SEQ ID NO: 23.

86. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 25, a heavy chain complementarity determining region 2 (CDR2) having an amino acid sequence at least 80% identical to SEQ ID NO: 26, and a heavy chain complementarity determining region 3 (CDR3) having an amino acid sequence at least 80% identical to SEQ ID NO: 27.

87. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 29, a heavy chain complementarity determining region 2 (CDR2) having an amino acid sequence at least 80% identical to SEQ ID NO: 30, and a heavy chain complementarity determining region 3 (CDR3) having an amino acid sequence at least 80% identical to SEQ ID NO: 31.

88. The method of claim 77, wherein the monoclonal antibody or antigen-binding fragment thereof comprises at least one of a heavy chain complementarity determining region 1 (CDR1) having an amino acid sequence at least 80% identical to SEQ ID NO: 33, a heavy chain complementarity determining region 2 (CDR2) having an amino acid sequence at least 80% identical to SEQ ID NO: 34, and a heavy chain complementarity determining region 3 (CDR3) having an amino acid sequence at least 80% identical to SEQ ID NO: 35.

89. The method of claim 77, wherein the monoclonal antibody or antigen-binding fragment thereof comprises a light chain variable domain (VL) comprising an amino acid sequence at least 80% identical to an amino acid sequence set forth as SEQ ID NO: 7, or SEQ ID NO: 15.

90. The method of claim 77, wherein the monoclonal antibody or antigen-binding fragment thereof comprises a heavy chain variable domain (VH) comprising an amino acid sequence at least 80% identical to an amino acid sequence set forth as SEQ ID NO: 8, SEQ ID NO: 16, SEQ ID NO: 20, SEQ ID NO: 24, SEQ ID NO: 28, SEQ ID NO: 32, SEQ ID NO: 36, or SEQ ID NO: 37.

91. The method of claim 77, wherein the monoclonal antibody or antigen-binding fragment thereof comprises a light chain variable domain (VL) comprising an amino acid sequence at least 80% identical to SEQ ID NO: 7 and a heavy chain variable domain (VH) comprising an amino acid sequence at least 80% identical to SEQ ID NO: 8.

92. The method of claim 77, wherein the monoclonal antibody or antigen-binding fragment thereof comprises a light chain variable domain (VL) comprising an amino acid sequence at least 80% identical to SEQ ID NO: 15 and a heavy chain variable domain (VH) comprising an amino acid sequence at least 80% identical to SEQ ID NO: 16.

93. The method of claim 77, wherein the monoclonal antibody or antigen-binding fragment thereof selectively binds to a complex comprising an HLA-E and a neoantigen.

94. The method of claim 77, wherein the monoclonal antibody or antigen-binding fragment thereof does not have a binding affinity to (i) the HLA-E alone; or (ii) the neoantigen alone.

95. The method of claim 77, wherein the neoantigen comprises, consisting essentially of, or consisting of a sequence according to SEQ ID NO: 38 (VMAPRTLFL).

96. The method of claim 77, wherein the complex comprises the HLA-E and VMAPRTLFL (SEQ ID NO: 38).

97. The method of claim 77, wherein the monoclonal antibody or antigen-binding fragment thereof is a murine antibody, a chimeric antibody, a camelid antibody, a humanized antibody, or a human antibody.

98. The method of claim 77, wherein the monoclonal antibody or antigen-binding fragment thereof is a TCR-like antibody.

99. The method of claim 77, wherein the monoclonal antibody or antigen-binding fragment thereof antibody further comprises a conjugated therapeutic moiety.

100. The method of claim 77, wherein the selective binding of the antibody to the complex comprising the HLA-E and the neoantigen induces an immune response in a cell.

101. The method of claim 100, wherein the immune response comprises activation of T cells.

102. The method of claim 101, wherein the T cell is a CD8+ T cell.

103. The method of claim 100, wherein the immune response comprises activation of cytotoxic T cells (CTLs).

104. The method of claim 77, wherein the antibody is administered continuously, at predetermined time intervals, or intermittently for 1, 2, 3, 4, 5, 6, 7, 8, 9, 10, 14, 15, 28, 30 or more days.

105. The method of claim 77, wherein the antibody is administered in 1 dose, 2 doses, 3 doses, 4 doses, 5 doses, 6 doses or more.

106. The method of claim 77, wherein the antibody is administered at a therapeutically effective amount.

107. The method of claim 77, wherein the cancer is breast cancer, kidney cancer, lung cancer, ovarian cancer, colorectal cancer, or a B-cell malignancy.
